(19) United States
(12) Patent Application Publication
(10) Pub. No.: US 2005/0272040 A1
(43) Pub. Date: Dec. 8, 2005

(54) Method for increasing synaptic growth or plasticity

(76) Inventor: Ira B. Black, Skillman, NJ (US)

Correspondence Address: LICATA & TYRRELL P.C., 66 EAST MAIN STREET, MARLTON, NJ, 08053, US

(21) Appl. No.: 10/533,355

(22) PCT Filed: Oct. 31, 2003
(86) PCT No.: PCT/US03/34777

§ 371 Date: Aug. 1, 2005

Related U.S. Application Data
(60) Provisional application No. 60/422,986, filed on Nov. 1, 2002.

(51) Int. Cl. C12Q 1/68; A61K 48/00; A61K 38/17

(52) U.S. Cl. 435/6; 514/2; 514/44

(57) ABSTRACT

The present invention relates to a method of modulating synaptic growth or plasticity by increasing the expression of genes found to be induced by BNDF. Such genes include c-fos proto-oncogene, early growth response protein 1, activity-regulated cytoskeletal associated, fos-related antigen 2, G1/S-specific cyclin D1, voltagegated potassium channel protein, sodium channel beta 1 subunit, secretogranin II precursor, somatostatin receptor 4, transmembrane receptor UNC5 homology, neuropeptide Y, VGF protein precursor and protein-tyrosine phosphatase 1B. Methods of identifying agents which modulate the expression of these genes and the use of such agents for treating a disease or condition associated with damaged or diseased synapses are also provided.

### BACKGROUND OF THE INVENTION

Neurotrophin-induced modifications of synaptic strength have been associated with mechanisms of learning and memory (Chao (2000) J. Neurosci. Res. 59:353-355; Tyler et al. (2002) Learn. Mem. 9:224-237). However, the molecular genetics of BDNF-induced plasticity is limited. Several studies have drawn links between BDNF, learning, transcription and translation. For example, endogenous BDNF as well as transcriptional activation are necessary for the maintenance phase of long-term potentiation (L-LTP) (Korte et al. (1998) Neuropharmacology 37:553-559; Chen et al. (1999) J. Neurosci. 19:7983-7990; Patterson et al. (2001) Neuron 32:123-140). In addition, BDNF and trkB may be required for the acquisition, consolidation and recall some types of information (Tyler et al. (2002) supra; Yamada et al. (2002) Life Sci. 70:735-744). Further, BDNF alone is sufficient to trigger a form of long-term synaptic potentiation in vivo (BDNF-LTP) (Ying et al. (2002) J. Neurosci. 22:1532-1540), the induction of which requires transcription (Messaoudi et al., 2002). The regulation and function of specific candidate genes in BDNF-LTP, L-LTP and learning paradigms have been demonstrated (Hevroni et al. (1998) J. Mol. Neurosci. 10:75-98; Guzowski et al. (2001) J. Neurosci. 21:5089-5098; Ying et al. (2002) supra). Although studies have employed transcriptional profiling to define panels of genes associated with learning (Luo et al. (2001) J. Mol. Neurosci. 17:397-404; Cavallaro et al. (2002) Neurochem. Res. 27:1201-1207; Donahue et al. (2002) Hippocampus 12:821-833), the identification of a catalog of genes associated with BDNF-induced plasticity in vitro, and the correlation of expression of these genes with learning in vivo has not been studied.

The regulated genes identified by transcriptional profiling serve as powerful tools to decipher mechanisms of both BDNF-induced transcription and plasticity. It has been shown that BDNF elicits differential electrophysiological responses in individual neurons (Levine et al. (1996) Brain Res. Mol. Brain Res. 38:300-303; Gottschalk et al. (1998) J. Neurosci. 18:6830-6839; Lessmann and Heumann (1998) Neuroscience 86:399-413; Schinder et al. (2000) Neuron 25:151-163; Thakker-Varia et al. (2001) J. Neurosci. 21:6782-6790). This phenomenon has been attributed to target specificity (Schinder et al. (2000) supra) and/or initial synaptic strength (Gottschalk et al. (1998) supra; Lessmann and Heumann (1998) supra; Berninger et al. (1999) Learn Mem. 6:232-242); however, these studies did not address the molecular mechanisms associated with distinctive single cell synaptic responses to BDNF.

Moreover, while the signal transduction pathways required for BDNF-modulated synaptic plasticity have been examined (Gottschalk et al. (1999) Learn. Mem. 6:243-256; Minichiello et al. (2002) Neuron 36:121-137; Mizuno et al. (2003) Mol. Psychiatry 8:217-224), the requirement for various kinase pathways in BDNF-induced transcription remains to be determined.

Increased transcription during the processes of learning in vivo suggests that gene products may be involved in the formation of new memories. Transcriptional analysis following different learning tasks has revealed regulation of genes previously implicated in synaptic function (Guzowski et al. (2001) supra; Luo et al. (2001) supra; Cavallaro et al. (2002) supra; Donahue et al. (2002) supra).

There are only a few studies delineating functional roles for neuronal genes identified by transcriptional profiling (Huh et al. (2000) Science 290:2155-2159). Differential display has been used in conjunction with electrophysiological analysis and gene-targeted mice to identify a requirement for Rab3A, a synaptic vesicle trafficking protein, in BDNF-induced plasticity (Thakker-Varia et al. (2001) supra).

### SUMMARY OF THE INVENTION

One aspect of the present invention is a method for modulating synaptic growth or plasticity. The method encompasses increasing the expression of a BDNF-inducible nucleic acid sequence, or activity of a protein encoded thereby, so that synaptic growth or plasticity is stimulated. Preferably, the nucleic acid sequence which is inducible by BDNF is c-fos proto-oncogene (SEQ ID NO:1); early growth response protein 1 (SEQ ID NO:2); activity-regulated cytoskeletal associated (SEQ ID NO:3); fos-related antigen 2 (SEQ ID NO:4); G1/S-specific cyclin D1 (SEQ ID NO:5); voltage-gated potassium channel protein (SEQ ID NO:6); sodium channel, beta 1 subunit (SEQ ID NO:7); secretogranin II precursor (SEQ ID NO:8); somatostatin receptor 4 (SEQ ID NO:9); transmembrane receptor UNC5 homology (SEQ ID NO:10); neuropeptide Y (SEQ ID NO:11); VGF protein precursor (SEQ ID NO:12); or protein-tyrosine phosphatase 1B (SEQ ID NO:13).

A nucleic acid sequence or protein of the invention is intended to include those disclosed herein (i.e., SEQ ID NO:1-13 and proteins encoded thereby) or any fragment, homolog, or ortholog which has the capacity to modulate synaptic growth or plasticity.

Another aspect of the present invention is a method for identifying an agent which increases synaptic growth or plasticity. The method involves contacting a test cell with an agent and detecting activation of c-fos proto-oncogene (SEQ ID NO:1); early growth response protein 1 (SEQ ID NO:2); activity-regulated cytoskeletal associated (SEQ ID NO:3); fos-related antigen 2 (SEQ ID NO:4); G1/S-specific cyclin D1 (SEQ ID NO:5); voltage-gated potassium channel protein (SEQ ID NO:6); sodium channel, beta 1 subunit (SEQ ID NO:7); secretogranin II precursor (SEQ ID NO:8); somatostatin receptor 4 (SEQ ID NO:9); transmembrane receptor UNC5 homology (SEQ ID NO:10); neuropeptide Y (SEQ ID NO:11); VGF protein precursor (SEQ ID NO:12); or protein-tyrosine phosphatase 1B (SEQ. ID NO:13) nucleic acid sequences in the test cell. An increase in the activation of said nucleic acid sequences in the test cell contacted with the agent relative to the activation of said nucleic acid sequences in a test cell not contacted with the agent is indicative that said agent increases synaptic growth or plasticity.

A further aspect of the invention is a method for treating a disease or condition associated with damaged or diseased synapses. The method encompasses administering an effective amount of an agent which activates c-fos proto-oncogene (SEQ ID NO:1); early growth response protein 1 (SEQ ID NO:2); activity-regulated cytoskeletal associated (SEQ ID NO:3); fos-related antigen 2 (SEQ ID NO:4); G1/S-specific cyclin D1 (SEQ ID NO:5); voltage-gated potassium channel protein (SEQ ID NO:6); sodium channel, beta 1 subunit (SEQ ID NO:7); secretogranin II precursor (SEQ ID NO:8); somatostatin receptor 4 (SEQ ID NO:9); transmembrane receptor UNC5 homology (SEQ ID NO:10); neuropeptide Y (SEQ ID NO:11); VGF protein precursor (SEQ ID NO:12); or protein-tyrosine phosphatase 1B (SEQ ID NO:13) nucleic acid sequences.

### BRIEF DESCRIPTION OF THE DRAWINGS

FIG. 1 shows that Arc expression and synaptic response to BDNF correlate at the single-cell level. FIG. 1A presents Arc expression in individual BDNF-treated cells following electrophysiological recordings and aRNA amplification. Arc transcription was measured by real-time RT-PCR and then normalized to internal GAPDH levels. The data are then expressed as a ratio of the average Arc expression in the seven control cells (expression ratio). FIG. 1B presents a correlation of Arc expression ratios to individual cell's synaptic BDNF responses. Average synaptic charge 3 to 5 minutes post-BDNF exposure expressed as a fold increase over baseline (−2 to 0 minute) was plotted against Arc expression ratios shown in FIG. 1A. Each circle represents one BDNF-treated cell. The cell number next to the circle corresponds with the cell number in FIG. 1A. Dashed line depicts a correlation coefficient indicating significance (r=0.66., p<0.005). The data are categorized into two groups of BDNF-treated cells: a cluster of non-responders (shaded) and a spread of responders (clear). FIG. 1C shows the average Arc expression ratios of the two populations of cells defined in FIG. 1B. Responders (white bar) show a significantly higher fold increase in Arc expression (average±se, n=7) compared to non-responders (shaded bar) (average±se, n=9) (* p<0.05, t-test).

FIG. 2 shows that VGF as well as immediate early genes are upregulated after training with trace eyeblink conditioning. RNA was obtained from the hippocampi of naïve rats, unpaired controls and trace conditioned rats following 800 trials (200 trials/day for 4 days) of paired stimuli and subjected to real-time RT-PCR for a number of genes identified by microarray. Bars represent average gene expression±se for naïve rats (n=5, white bar), unpaired controls (n=3, stippled bar) and trace conditioning (n=8, black bar). All data is normalized to internal GAPDH levels and then expressed as a fold change over the amount of mRNA in naïve (control) rats. A number of immediate early genes including c-fos, EGR1, and Arc were upregulated after trace conditioning. In addition, the secreted polypeptide VGF was increased following paired trace conditioning. *indicates paired samples are significantly different from unpaired and naive control samples (p<0.05, ANOVA).

### DETAILED DESCRIPTION OF THE INVENTION

Whole-cell patch-clamp recordings in conjunction with differential display and single-cell transcriptional analysis were used to identify known and novel genes with altered expression in BDNF-induced plasticity. The functions of some of the identified genes have been defined using various methods.

The molecular genetics underlying BDNF-evoked plasticity were examined by expression profiling using cDNA microarrays on a population of hippocampal cells in vitro. Synaptic effects of BDNF are observed within 20 minutes of exposure (Levine et al. (1996) supra; Lessmann and Heumann (1998) supra; Schinder et al. (2000) supra). A 3-hour BDNF treatment has been used to identify several genes with roles in synaptic plasticity (Thakker-Varia et al. (2001) supra). Consequently, to reveal immediate, as well as downstream transcriptional changes, both acute (20 minutes) and longer treatment (3 hours) with BDNF were used herein. Approximately 8% of the arrayed cDNAs exhibited differential expression at 20 minutes and 3 hours. Genes involved in synaptic plasticity and exhibiting an increase of 1.7-fold over control were selected for confirmation by real-time reverse transcription PCR (RT-PCR). Table 1 lists the genes induced by BDNF treatment. Two independent Atlas arrays were performed using RNA from hippocampal cells treated for either 20 minutes or 3 hours of BDNF (50 ng/mL) and control sister cultures.

TABLE 1

| | | | | | |
|---|---|---|---|---|---|
| Clone | Gene/Protein | Accession | | | SEQ ID |
| ID | Name | Number | Fold Change | NGF | NO: |

20 Minute BDNF Treatment

| | | | | | |
|---|---|---|---|---|---|
| A10g | c-fos | X06769 | 2.2 ± 0.4 | UC | 1 |
| E06d | EGR1 | M18416 | 4.3 ± 0.8 | UC | 2 |
| F13I | Arc | U19866 | 13.7 ± 5.0 | UC | 3 |

3 Hour BDNF Treatment

| | | | | | |
|---|---|---|---|---|---|
| A05d | Fra2 | U18913 | 2.3 ± 0.4 | DOWN | 4 |
| A07a | CyclinD1 | D14014 | 3.3 ± 0.5 | UC | 5 |
| A10g | c-fos | X06769 | 2.3 ± 0.4 | UC | 1 |
| B05d | Kv + 1.1 | M26161 | 1.4 ± 0.1 | UC | 6 |
| B06j | Na + ch | M91808 | 1.8 ± 0.2 | UC | 7 |
| B13n | SgII | M93669 | 5.1 ± 1.2 | UC | 8 |
| D04i | SSTR4 | U04738 | 1.7 ± 0.1 | DOWN | 9 |
| D11k | UNC5H2 | U87306 | 2.2 ± 0.6^{†} | UC | 10 |
| D13l | NPY | M20373 | 1.9 ± 0.3^{†} | DOWN | 11 |
| D14f | VGF | M60525 | 6.5 ± 1.5 | UC | 12 |
| E06d | EGR1 | M18416 | 6.6 ± 0.9 | UC | 2 |
| E12f | PTP1B | M33962 | 1.4 ± 0.1 | UC | 13 |
| F13l | Arc | U19866 | 34.8 ± 4.3 | UC | 3 |

The criteria for selection from the array was a >1.7-fold change. RT-PCR validated the induction of 16 genes (average ± standard error, n = 6, ^{†}n = 5, p < 0.05, t-test).
UC = unchanged, down = decreased.

Of those genes selected, the regulation of 7% at 20 minutes and 40% at 3 hours were validated, although the magnitude of regulation was not always identical to that observed on the microarray. To focus on genes specifically involved in plasticity, a related neurotrophin, nerve growth factor (NGF), not inducing synaptic plasticity (Levine et al. (1996) supra), served as a control. NGF did not affect transcription of most genes, indicating the specificity of BDNF.

Genes upregulated by BDNF were categorized into different functional classes. At 20 minutes, immediate early genes were upregulated, whereas at 3 hours, in addition to those same immediate early genes, channel proteins, receptors and neuropeptides exhibited increases. Two genes were of particular interest due to their association with synaptic plasticity: Arc/Arg 3.1, an effector of immediate early genes and VGF, a secreted polypeptide located in large dense core vesicles (Salton et al. (2000) Front. Neuroendocrinol. 21:199-219). Arc, the most highly regulated gene at both 20 minutes and 3 hours, has been implicated in BDNF-induced plasticity and LTP (Guzowski et al. (2000) J. Neurosci. 20:3993-4001; Messaoudi et al. (2002) J. Neurosci. 22:7453-7461; Steward and Worley (2002) Neurobiol. Learn. Mem. 78:508-527; Ying et al. (2002) supra). VGF, previously cloned from plate V of a NGF-induced PC12 cDNA library (Salton et al. (2000) supra), in contrast, was only increased at 3 hours and has not been associated with BDNF-induced synaptic efficacy. These two genes were used in subsequent experiments as indices of synaptic efficacy and prototypes for functional studies.

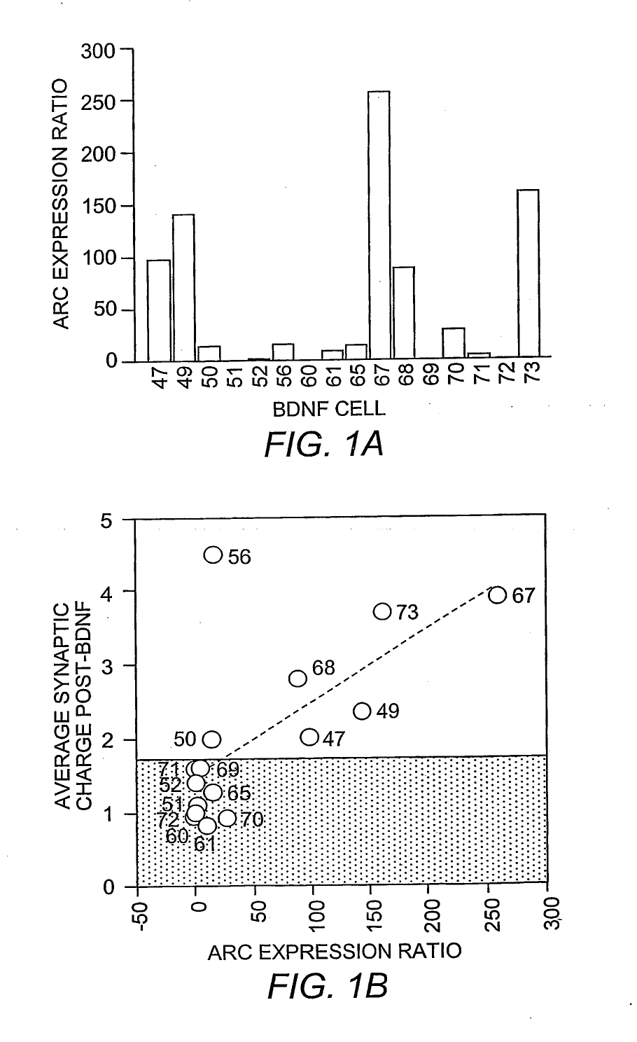

To examine the relationship between synaptic response and gene expression, an individual cell's electrophysiological response to BDNF was examined in conjunction with single-cell RT-PCR for Arc. Previous studies revealed a range of synaptic activities within a population of pyramidal-like hippocampal neurons following 3 hours BDNF exposure (Thakker-Varia et al. (2001) supra). Whole-cell patch-clamp recordings from 16 cells verified 0.8- to 4.5-fold increase in synaptic charge following 20 minutes BDNF exposure. Average synaptic charge for control cells not exposed to BDNF (n=7) exhibited no change in synaptic charge during the length of the recording. A group of BDNF-treated cells presenting a minimal synaptic response was apparent. This group was categorized as non-responders (FIG. 1B) and the other cells showing a greater response to BDNF were classified as responders. To determine whether cellular basal activity influences the response to BDNF, the average synaptic charge pre-BDNF of the responders (36±13.1 pC, n=7) versus the non-responders (23.2±6.6 pC, n=9) was compared. There was no statistical difference in the populations (p>0.05, t-test) examined between initial synaptic strength and responsiveness to BDNF, indicating that this parameter is not responsible for the differential response.

The molecular genetics of differential synaptic responses to BDNF was examined by performing single-cell transcriptional analysis after whole-cell patch-clamp. Arc was selected as a prototype due to its acute and robust induction by BDNF in a population of hippocampal neurons. To assess the relationship between differential electrophysiological responses to BDNF and Arc gene expression, individual cells were harvested following electrophysiological recordings. FIG. 1A represents Arc expression in cells exposed to BDNF relative to the average Arc expression in cells not exposed to BDNF (control) as assessed by real-time RT-PCR (expression ratio). A range of Arc expression levels was apparent within the group of 16 BDNF-treated cells, indicating transcriptional differences among pyramidal-like hippocampal cells.

The relationship between the synaptic response to BDNF and transcriptional changes was examined. Synaptic charge was plotted against Arc expression ratios for individual cells (FIG. 1B). A linear correlation (r=0.66, p<0.005) was evident as depicted by the dashed line, indicating a direct relationship between synaptic plasticity and gene expression. Alternatively, the data may be viewed as representing two sets of BDNF-treated cells: a cluster of non-responders (shaded area) and a spread of responders (clear area). Classification of cells into electrophysiologic responders or non-responders, revealed a striking difference in Arc expression ratios (FIG. 1C). Responders exhibit a 17-fold increase in Arc expression relative to the non-responders (p<0.05, t-test). These results demonstrate that the magnitude of BDNF-induced synaptic plasticity is reflected in gene expression in individual hippocampal cells. Enhanced transcription following neurotrophin exposure, may represent a mechanism for long-term maintenance of increased synaptic activity as has been shown for L-LTP (Nguyen et al. (1994) Science 265:1104-1107).

Signaling cascades involved in BDNF-mediated synaptic strengthening include mitogen-activated protein (MAP) kinase, phospholipase C-γ (PLC-γ) and phosphoinositide-3 kinase (PI3K) (Gottschalk et al. (1999) supra; Minichiello et al. (2002) supra; Ying et al. (2002); Mizuno et al. (2003) supra). To determine whether these transduction pathways are also required for BDNF transcriptional regulation of immediate early genes at 20 minutes and for VGF at 3 hours, pharmacological inhibitors were employed (Table 2).

TABLE 2

| Inhibitor | 20 Minutes | | | 3 Hours |
|---|---|---|---|---|
| (Protein Inhibited) | c-fos | EGR-1 | Arc | VGF |
| K252a (Tyr Kinase) | Blocked | Blocked | Blocked | Blocked |
| U0126 (MEK) | Blocked | Blocked | Blocked | Blocked |
| PD98059 (MEK) | Blocked | Blocked | Blocked | Blocked |
| U73122 (PLC-γ) | Blocked | UC | Blocked | Blocked |
| LY294002 (PI3 Kinase) | UC | UC | UC | UC |
| KN93 (CaMKII) | Blocked | Blocked | Blocked | Blocked |
| H-89 (PKA) | UC | UC | UC | UC |
| CC (PKC) | UC | UC | UC | UC |

Effect of inhibitors on BDNF-induced gene transcription. The regulation of BDNF-induced genes in the presence of inhibitors was determined by real time RT-PCR. Neurons were pre-incubated with inhibitors for 30 minutes prior to BDNF treatment (20 minutes for immediate early genes and 3 hours for VGF). One group of cells in each experiment was treated with inhibitor alone and showed no significant change in expression relative to vehicle control. “blocked” indicates significant reduction of transcription in the presence of inhibitor plus BDNF relative to BDNF alone (p<0.05, ANOVA, n=3). UC (unchanged) indicates no significant effect of inhibitor on transcription.

Each experiment consisted of four groups: vehicle, inhibitor alone, BDNF, BDNF plus inhibitor. Data were normalized to GAPDH and expressed as a fold change of vehicle control. Inhibitor alone showed no significant effect on transcription. K252a, U0126, PD98059, U73122 and KN93 blocked BDNF-induced transcription of the immediate early genes and VGF whereas LY294002, H-89 and chelerythrine chloride (CC) did not affect gene expression. K252a blocked the BDNF effect, indicating that transcription results from trkB stimulation. Pretreatment with either MAP kinase inhibitor (U0126 or PD98059) blocked BDNF-induced transcription, indicating a requirement for MEK-mediated phosphorylation of MAP kinase. The PLC-γ inhibitor (U73122) and the Ca2+/calmodulin-dependent protein kinase (CaMK) inhibitor (KN93) blocked transcription of most immediate early genes and VGF (Table 2). In contrast, a number of signaling pathways were not involved in BDNF-induced transcription, shown by the lack of effect of inhibitors of PI3K (LY294002), protein kinase A (PKA) (H-89) and protein kinase C (PKC) (chelerythrine chloride, CC). These results indicate that BDNF-mediated transcription at both 20 minutes and 3 hours was dependent on the CaMK and PLC-γ pathways in addition to trkB and MAP kinase.

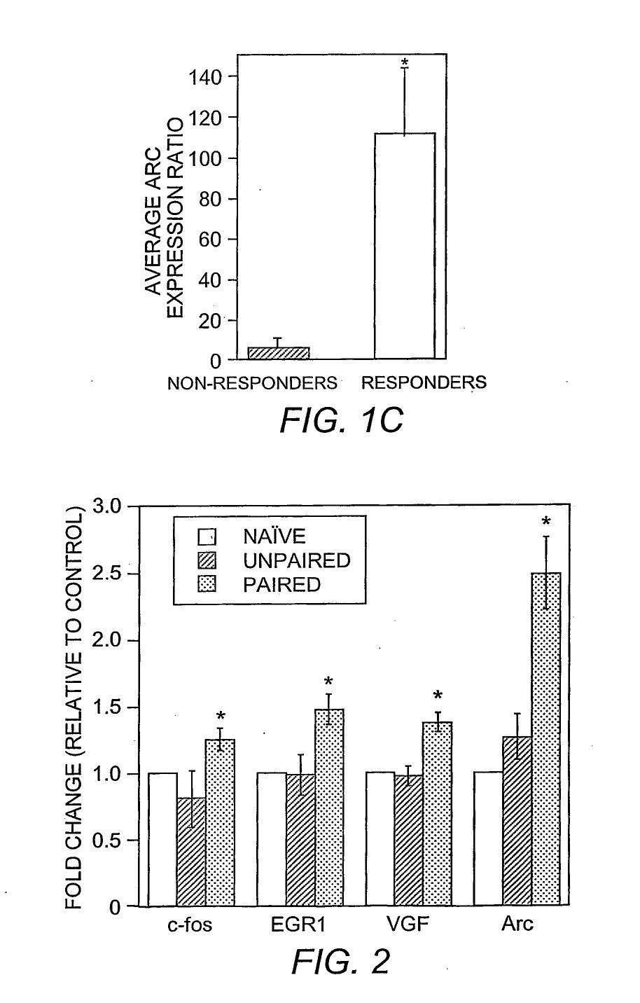

To determine whether VGF and other transcripts induced by BDNF in vitro predict alterations in mRNA in vivo, gene expression was examined following training on a hippocampal-dependent learning task. Rats were exposed to 800 trials of either paired or unpaired trace eyeblink conditioning and sacrificed 24 hours later. Eyeblinks were assessed by eyelid electromyography and those occurring during the 500 ms trace interval were considered conditioned responses (CRs). Rats exposed to paired stimuli exhibited 60% (±4) CRs, whereas those exposed to the same number of stimuli presented in an explicitly unpaired manner exhibited 11% (±1) CRs, a percentage similar to that of spontaneous blinks (10%). A t-test for independent samples on the number of conditioned responses revealed that the difference was significant at p<0.0001. VGF transcripts were significantly upregulated in the hippocampus after paired but not unpaired eyeblink conditioning or naive controls (p<0.05, ANOVA) (FIG. 2). Several of the immediate early genes including c-fos, EGR1 and Arc were also induced by exposure to the paired stimuli (p<0.05, ANOVA). These changes were specific and selective, since other genes induced by BDNF in vitro were not upregulated by trace eyeblink conditioning; Na+ channel, somatostatin receptor 4 (SSTR4) and transmembrane receptor UNC homolog 2 (UNC5H2) were not significantly enhanced following paired eyeblink training. These results indicate that BDNF-induced transcriptional alterations of VGF and immediate early genes in vitro predict changes in vivo in the hippocampus during memory formation. Accordingly, modulating the neuropeptide VGF, for example, is useful for regulating synaptic function associated with learning and memory.

The products of the genes induced following learning may regulate neuronal plasticity at the single-cell level. VGF, like BDNF, is a secreted polypeptide and thus may be relevant for intercellular communication. A functional role for VGF in synaptic plasticity was thus investigated. To determine whether BDNF-induced transcriptional changes of VGF were accompanied by translational alterations, VGF levels were examined following BDNF treatment by western blot analysis. VGF, migrating as a doublet with an apparent molecular weight of 85-90 kD, increased by 1.5-fold within 3 hours of BDNF exposure. VGF protein levels were maximal at 12 hours and remained elevated for 48 hours of continuous BDNF application. Consequently, increased VGF mRNA levels after 3 hours BDNF treatment were associated with enhanced protein translation. VGF protein processing occurs in both neurons and neuroendocrine cells (Trani et al. (1995) J. Neurochem. 65:2441-2449; Salton et al. (2000) supra). The localization of VGF in dissociated hippocampal cultures was examined using a C-terminal antibody (R-15) which labeled both the soma and neurites indicating translocation to the neuronal processes. The expression of VGF was similar to that previously observed in primary neuronal cultures (Benson and Salton (1996) Brain Res. Dev. Brain Res. 96:219-228) and was punctate in appearance. This pattern is consistent with the regulated release of VGF from large dense core vesicles (Trani et al. (1995) supra; Benson and Salton (1996) supra; Possenti et al. (1999) Endocrinology 140:3727-3735) and indicates a physiological role for the peptide.

To explore a functional role for VGF in neuromodulation, whole-cell patch-clamp recordings on hippocampal cells were performed in the presence of synthetic VGF peptides. The electrophysiological profile of VGF peptides on hippocampal cells was similar to that observed for BDNF (Levine et al. (1996) supra). Increased synaptic charge was observed within 5 minutes of perfusion of peptides derived from the C-terminal portion of VGF (Thr-Leu-Gln-Pro-62; SEQ ID NO:14), whereas no change in synaptic charge resulted from application of peptide from the N-terminal region (Leu-Glu-Gly-Ser-28; SEQ ID NO:15). Enhanced synaptic activity was maintained for up to 25 minutes in the continued presence of Thr-Leu-Gln-Pro-62 (SEQ ID NO:14). As an independent confirmation of the effect of VGF on synaptic plasticity, an alternate peptide derived from the C-terminal region was tested (Ala-Gln-Glu-Glu-30; SEQ ID NO:16) and revealed a similar effect on synaptic charge. The effects of both Ala-Gln-Glu-Glu-30 (SEQ ID NO:16) and Thr-Leu-Gln-Pro-62 (SEQ ID NO:14) peptides were dose-dependent, saturating at 0.1 μM. Taken together, these data indicate that the functional portion of VGF resides in the C-terminal region of the protein. These findings are the first demonstration of a neuromodulatory role for VGF in synaptic plasticity of the hippocampus which subserves learning and memory.

The present invention relates to differentially-expressed genes involved in BDNF regulation of synaptic activity. These genes, referred to herein as nucleic acid sequences of the invention, which were inducible by BNDF include c-fos proto-oncogene (c-fos; SEQ ID NO:1); early growth response protein 1 (EGR1; SEQ ID NO:2); activity-regulated cytoskeletal associated (Arc; SEQ ID NO:3); fos-related antigen 2 (Fra2; SEQ ID NO:4); G1/S-specific cyclin D1 (cyclinD1; SEQ ID NO:5); voltage-gated potassium channel protein (Kv+ 1.1; SEQ ID NO:6); sodium channel, beta 1 subunit (Na+ ch; SEQ ID NO:7); secretogranin II precursor (SgII; SEQ ID NO:8); somatostatin receptor 4 (SSTR4; SEQ ID NO:9); transmembrane receptor UNC5 homology (UNC5H2; SEQ ID NO:10); neuropeptide Y (NPY; SEQ ID NO:11); VGF protein precursor (VGF; SEQ ID NO:12); and protein-tyrosine phosphatase 1B (PTP1B; SEQ ID NO:13). Said genes were found to be upregulated or activated in BDNF-induced plasticity. Accordingly, as these genes may contribute to synaptic growth and plasticity, agents which modulate the expression or activity of these genes are useful for modulating learning and memory. As used herein, synaptic growth and plasticity is intended to include both acute and long-term activation, presynaptic and postsynaptic, and basal and enhanced transmission.

Accordingly, one aspect of the present invention is a method for increasing synaptic growth or plasticity via activating or increasing the expression of a nucleic acid sequence, which is inducible by BNDF, or the activity of a protein encoded thereby. Such an increase in gene expression or protein activity is useful in promoting or stimulating synaptic growth or plasticity to promote or stimulate, for example, memory or learning. Means of increasing the expression of a nucleic acid sequence disclosed herein or the activity of proteins encoded thereby are provided.

It is contemplated that the expression of a BNDF-induced nucleic acid sequence of the invention may be increased via genetic engineering (gene therapy) or by administering agents which increase the expression or activity of said nucleic acid sequences or proteins encoded thereby, respectively. It is further contemplated that a protein of the invention may be administered as a purified recombinant protein (e.g., encapsulated in liposome formulations) to increase the amount of said protein in a cell. In particular it is advantageous for the gene, agent or protein to be expressed or administered to cells of the brain, e.g., the hippocampus.

In one embodiment, genetic engineering or gene therapy approaches to increase the expression of a nucleic acid sequence of the invention to promote learning or memory may employ any method known in the art. For example, a method for increasing expression of a nucleic acid sequence of the invention includes exogenously supplying said nucleic acid sequence to a cell (i.e., a gene knockin).

In general, the coding sequence of a nucleic acid sequence of the invention (i.e., mRNA or DNA sequence) is delivered to cells in an area in which increased memory or learning is desirable. Mechanical methods; such as microinjection, liposome-mediated transfection, electroporation, or calcium phosphate precipitation, may be used. Alternatively, if it is desired that the cells stably retain the construct, it may be supplied on a plasmid and maintained as a separate element or integrated into the genome of the cells, as is known in the art. Additionally, the construct may be introduced into a neuronal cell (e.g., cortical neurons or dorsal root ganglion neurons) or a cell capable of differentiating into a neuronal cell (e.g., stem cell) and be transplanted into an individual in need. The construct may include transcriptional regulatory elements, such as a promoter element, an enhancer or UAS element, and a transcriptional terminator signal, for controlling transcription of the coding sequence in the cells.

A DNA construct or RNA molecule encoding a nucleic acid sequence of the invention may be directly introduced into a cell using well-known methods or incorporated into a viral vector for delivery to the host animal. Vectors, such as replication-defective retroviruses, rabies virus, adenoviruses and adeno-associated viruses may be used, preferably a neurotropic virus is used. Protocols for producing recombinant viruses and for infecting cells in vitro or in vivo with such viruses are described by in Ruitenberg, et al. ((2002) Methods 28(2):182-94); Lundstom ((2001) Curr. Gene Ther. 1(1):19-29) and Current Protocols in Molecular Biology, Ausubel, F. M. et al. (eds.) Greene Publishing Associates, (1989), Sections 9.10-9.14 and other standard laboratory manuals. The genome of an adenovirus may be manipulated such that it encodes and expresses a nucleic acid sequence of the invention but is inactivated in terms of its ability to replicate in a normal lytic viral life cycle (Berkner, et al. (1988) BioTechniques 6:616; Rosenfeld, et al. (1991) Science 252:431-434; Rosenfeld, et al. (1992) Cell 68:143-155). Suitable adenoviral vectors derived from the adenovirus strain Ad type 5 dl324 or other strains of adenovirus (e.g., Ad2, Ad3, Ad7, etc.) are well-known to those skilled in the art. In vivo use of adenoviral vectors is described in Flotte, et al. ((1993) Proc. Natl. Acad. Sci. USA 90:10613-10617) and Kaplitt, et al. ((1994) Nature Genet. 8:148-153). Other viral vectors, such as those based on togaviruses, alpha viruses, or vaccinia virus may also be used. Alternatively, an adeno-associated virus vector such as that disclosed by Xu, et al. ((2001) Gene Ther. 8(17):1323-32) may be used to express a nucleic acid sequence of the invention.

Regulatory sequences for in vivo expression of a nucleic acid sequence of the invention are preferably brain-specific and may be constitutive or regulated. Exemplary regulatory sequences include those associated with the expression of prions, human glial fibrillary acidic protein gene (Shi, et al. (2001) Proc. Natl. Acad. Sci. USA 98(22):12754-9), neuron-specific enolase or beta-actin.

A DNA construct or RNA molecule of the invention may also be combined with a condensing agent, such as polylysine, polyarginine, polyornithine, protamine, spermine, spermidine, or putrescine, to form a gene delivery vehicle. Many suitable methods for making such linkages are known in the art. Alternatively, a DNA construct or RNA molecule may be associated with a liposome for delivery to a desired cell. For example, plasmid DNA may be targeted to the brain with pegylated immunoliposomes using a targeting ligand such as a peptidomimetic mAb, which binds to a transporting receptor on the blood-brain barrier (Shi, et al. (2001) Proc. Natl. Acad. Sci. USA 98(22):12754-9). Other suitable methods of providing DNA constructs or RNA molecules include DNA-ligand combinations. Receptor-mediated DNA delivery techniques are also well-known in the art (Findeis, et al. (1993) Trends Biotech. 11:202-05; Chiou, et al. (1994) Gene Therapeutics: Methods and Applications of Direct Gene Transfer (J. A. Wolff, ed.); Wu, et al. (1994) J. Biol. Chem. 269:542-46). Expression of a nucleic acid sequence of the invention may be monitored by detecting production of mRNA which hybridizes to a delivered coding sequence or by detecting the protein product of the gene using, for example, immunological techniques.

Expression of an endogenous nucleic acid sequence of the invention in a cell may also be altered by introducing in-frame with the endogenous gene a DNA construct comprising a targeting sequence, a regulatory sequence, an exon, and an unpaired splice donor site by homologous recombination, such that a homologously recombinant cell comprising a new transcription unit is formed. The new transcription unit may be used to turn expression of a nucleic acid sequence of the invention on or off as desired. This method of affecting endogenous gene expression is taught in U.S. Pat. No. 5,641,670.

Alternatively, expression of a nucleic acid sequence of the invention may be altered in cells which have been removed from a mammal, such as neuronal cells. The cells may then be replaced into the same or another mammal, preferably to or within the vicinity of a region which learning or memory is to be increased.

In another embodiment, a recombinantly-produced or chemically-synthesized protein of the invention may be used to increase said protein levels in a cell where it is desirable to increase or stimulate learning or memory. Proteins of the invention may be used in a pharmaceutically acceptable composition and may be administered intraperitoneally, intracranially, subcutaneously, in accordance with well-known protocols.

As will be appreciated by one of skill in the art, a full-length protein of the invention may be produced for use in the methods of the invention, however, fragments of a protein of the invention may also be used provided the fragment maintains the desired binding interaction or activity of the full-length protein. Exemplary fragments of VGF which may be used to modulate synaptic growth or plasticity include fragments from the C-terminal region of VGF, e.g., Ala-Gln-Glu-Glu-30 (SEQ ID NO:16) and Thr-Leu-Gln-Pro-62 (SEQ ID NO:14) peptides.

In general, recombinant production of a protein of the invention requires incorporation of nucleic acid sequences encoding said protein (e.g., SEQ ID NO:1-13) into a recombinant expression vector in a form suitable for expression of the protein in a host cell.

A suitable form for expression provides that the recombinant expression vector includes one or more regulatory sequences operatively-linked to a nucleic acid sequence encoding a protein of the invention in a manner which allows for transcription of the nucleic acids into mRNA and translation of the mRNA into the protein. Regulatory sequences may include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector may depend on such factors as the choice of the host cell to be transfected and/or the level of expression required. Nucleic acid sequences or expression vectors harboring nucleic acid sequences encoding a protein of the invention may be introduced into a host cell, which may be of eukaryotic or prokaryotic origin, by standard techniques for transforming cells. Suitable methods for transforming host cells may be found in Sambrook, et al. (Molecular Cloning: A Laboratory Manual, 3rd Edition, Cold Spring Harbor Laboratory Press (2000)) and other laboratory manuals. The number of host cells transformed with a nucleic acid sequence encoding a protein of the invention will depend, at least in part, upon the type of recombinant expression vector used and the type of transformation technique used. Nucleic acids may be introduced into a host cell transiently, or more typically, for long-term expression of a protein of the invention the nucleic acid sequence is stably integrated into the genome of the host cell or remains as a stable episome in the host cell. Once produced, a protein of the invention may be recovered from culture medium as a secreted polypeptide, although it also may be recovered from host cell lysates when directly expressed without a secretory signal. When a protein of the invention is expressed in a recombinant cell other than one of human origin, said protein is substantially free of proteins or polypeptides of human origin. However, it may be necessary to purify the protein from recombinant cell proteins or polypeptides using conventional protein purification methods to obtain preparations that are substantially homogeneous as to a protein of the invention. As a first step, the culture medium or lysate is centrifuged to remove particulate cell debris. The membrane and soluble protein fractions are then separated. The recombinant protein may then be purified from the soluble protein fraction. The recombinant protein thereafter is purified from contaminant soluble proteins and polypeptides using any of the following suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, SEPHADEX G-75; ligand affinity chromatography, and protein A SEPHAROSE columns to remove contaminants such as IgG.

In addition to recombinant production, a protein of the invention may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer, Boston, Mass.). Various fragments of a protein of the invention may be chemically-synthesized separately and combined using chemical methods to produce a full-length molecule.

In a further embodiment of the present invention, expression of a nucleic acid sequence of the invention or activity of a protein encoded thereby may be regulated using an agent which increases the expression or activity of a protein of the invention. Agents suitable for regulating the expression or activity of protein of the invention may be identified in accordance with the screening assay of the invention and encompass numerous chemical classes, though typically they are organic molecules, preferably small organic compounds having a molecular weight of more than 100 and less than about 2,500 daltons. Agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents may also be found among biomolecules including peptides, antibodies, saccharides, fatty acids, steroids, purines, pyrimidines, derivatives, or proteins, DNA constructs or RNA molecules described herein and structural analogs or combinations thereof.

Agents are obtained from a wide variety of sources including libraries of synthetic or natural compounds. The use of replicable genetic packages, such as the bacteriophages, is one method of generating novel polypeptide entities that regulate the expression or activity of a protein of the invention. This method generally consists of introducing a novel, exogenous DNA segment into the genome of a bacteriophage (or other amplifiable genetic package) so that the polypeptide encoded by the non-native DNA appears on the surface of the phage. When the inserted DNA contains sequence diversity, then each recipient phage displays one variant of the template amino acid sequence encoded by the DNA, and the phage population (library) displays a vast number of different but related amino acid sequences.

Antibodies which specifically bind a protein of the invention are also contemplated as agonistic agents for regulating the activity of a protein of the invention. Antibodies to a protein of the invention may be generated using methods that are well-known in the art. Such antibodies may include, but are not limited to, polyclonal, monoclonal, chimeric, single chain, Fab fragments, and fragments produced by a Fab expression library. Neutralizing antibodies are especially preferred for therapeutic use.

For the production of antibodies, various hosts including goats, rabbits, rats, mice, humans, and others, may be immunized by injection with a protein of the invention or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to a protein of the invention have an amino acid sequence consisting of at least five amino acids and more preferably at least 10 amino acids. It is also preferable that they are identical to a portion of the amino acid sequence of the natural protein, and they may contain the entire amino acid sequence of a small, naturally occurring molecule. Short stretches of amino acids of a protein of the invention may be fused with those of another protein such as keyhole limpet hemocyanin and antibody produced against the chimeric molecule.

Monoclonal antibodies to a protein of the invention may be prepared using any technique which provides for the production of antibody molecules by continuous cell lines in culture. These include, but are not limited to, the hybridoma technique, the human B-cell hybridoma technique, and the EBV-hybridoma technique (Kohler, et al. (1975) Nature 256:495-497; Kozbor, et al. (1985) J. Immunol. Methods 81:31-42; Cote, et al. (1983) Proc. Natl. Acad. Sci. 80:2026-2030; Cole, et al. (1984) Mol. Cell Biol. 62:109-120).

In addition, techniques developed for the production of humanized and chimeric antibodies, the splicing of mouse antibody genes to human antibody genes to obtain a molecule with appropriate antigen specificity and biological activity may be used (Morrison, et al. (1984) Proc. Natl. Acad. Sci. 81, 6851-6855; Neuberger, et al. (1984) Nature 312:604-608; Takeda, et al. (1985) Nature 314:452-454). Alternatively, techniques described for the production of single chain antibodies may be adapted, using methods known in the art, to produce specific single chain antibodies. Antibodies with related specificity, but of distinct idiotypic composition, may be generated by chain shuffling from random combinatorial immunoglobulin libraries (Burton (1991) Proc. Natl. Acad. Sci. 88,11120-11123).

Antibodies may also be produced by inducing in vivo production in the lymphocyte population or by screening immunoglobulin libraries or panels of highly specific binding reagents as is well-known in the art (Orlandi, et al. (1989) Proc. Natl. Acad. Sci. 86: 3833-3837; Winter, et al. (1991) Nature 349:293-299).

Antibody fragments, which contain specific binding sites for a protein of the invention, may also be generated. For example, such fragments include, but are not limited to, the F(ab′)₂ fragments which may be produced by pepsin digestion of the antibody molecule and the Fab fragments which may be generated by reducing the disulfide bridges of the F(ab′)₂ fragments. Alternatively, Fab expression libraries may be constructed to allow rapid and easy identification of monoclonal Fab fragments with the desired specificity (Huse, et al. (1989) Science 254:1275-1281).

Various immunoassays may be used for screening to identify antibodies having the desired specificity. Numerous protocols for competitive binding or immunoradiometric assays using either polyclonal or monoclonal antibodies with established specificity are well-known in the art. Such immunoassays typically involve the measurement of complex formation between a specific antibody and a protein of the invention. A two-site, monoclonal-based immunoassay utilizing monoclonal antibodies reactive to two non-interfering epitopes is preferred, but a competitive binding assay may also be employed.

While isolated proteins, DNA constructs, RNA molecules, or antibodies disclosed heretofore may be used for increasing the expression or activity of a protein of the invention so that synaptic growth or plasticity is modulated, the present invention also provides a method for identifying other agents for such purposes. A cell-based assay is specifically contemplated. Such a method involves contacting a test cell with an agent and detecting expression or activation of a nucleic acid sequence of the invention in the test dell. An agent which causes an increase in activation of a nucleic acid sequence of the invention in the test cell when compared to a test cell not contacted with the agent, indicates that the agent increases synaptic growth or plasticity.

One embodiment of the screening method of the invention is based on the use of a reporter to detect gene expression. In this embodiment, test cells are typically isolated neurons which contain or have been transformed with a nucleic acid sequence encoding a reporter operably linked to promoter of a nucleic acid sequence of the invention. Upon exposure to the test agent, the presence or activity of the reporter is detected and correlated with the expression or activation of a nucleic acid sequence of the invention.

A nucleic acid sequence encoding a reporter may be inserted into a recombinant expression vector to generate a reporter gene construct. A reporter gene construct refers to a plasmid, virus or other vehicle known in the art that has been manipulated by insertion or incorporation of nucleic acid sequences encoding a reporter. Such reporter gene constructs of the invention are preferably plasmids which contain at least a portion of a promoter sequence of a nucleic acid sequence of the invention which is operably associated with the inserted nucleic acid sequences encoding the reporter. The construct typically contains an origin of replication as well as specific selectable or screenable marker genes for initially isolating, identifying or tracking test cells that contain the desired reporter/promoter DNA. The reporter gene construct also may provide unique or conveniently located restriction sites to allow severing and/or rearranging portions of the DNA inserts in a reporter gene construct. More than one reporter gene may be inserted into the construct such that the test cells containing the resulting construct may be assayed by different means.

A reporter refers to any sequence that is detectable and distinguishable from other sequences present in test cells. Preferably, the reporter nucleic acid sequence encodes a protein that is readily detectable either by its presence, or by its activity that results in the generation of a detectable signal. A nucleic acid sequence encoding the reporter is used in the invention to monitor and report the activity of a promoter of a nucleic acid sequence of the invention in test cells.

A variety of enzymes may be used as reporters including, but are not limited to, β-galactosidase (Nolan, et al. (1988) Proc. Natl. Acad. Sci. USA 85:2603-2607), chloramphenicol acetyltransferase (CAT; Gorman, et al. (1982) Mol. Cell Biol. 2:1044; Prost, et al. (1986) Gene 45:107-111), β-lactamase, β-glucuronidase and alkaline phosphatase (Berger, et al. (1988) Gene 66:1-10; Cullen, et al. (1992) Meth. Enzymol. 216:362-368). Transcription of the nucleic acid sequences encoding a reporter leads to production of the enzyme in test cells. The amount of enzyme present may be measured via its enzymatic action on a substrate resulting in the formation of a detectable reaction product. The method of the invention provides means for determining the amount of reaction product, wherein the amount of reaction product generated or the remaining amount of substrate is related to the amount of enzyme activity. For some enzymes, such as β-galactosidase, β-glucuronidase and β-lactamase, well-known fluorogenic substrates are available that allow the enzyme to convert such substrates into detectable fluorescent products.

A variety of bioluminescent, chemiluminescent and fluorescent proteins also may be used as light-emitting reporters. Exemplary light-emitting reporters, which are enzymes and require cofactor(s) to emit light, include, but are not limited to, the bacterial luciferase (luxAB gene product) of Vibrio harveyi (Karp (1989) Biochim. Biophys. Acta 1007:84-90; Stewart, et al. (1992) J. Gen. Microbiol. 138:1289-1300), and the luciferase from firefly, Photinus pyralis (De Wet, et al. (1987) Mol. Cell. Biol. 7:725-737).

Another type of light-emitting reporter, which does not require substrates or cofactors includes, but is not limited to, the wild-type green fluorescent protein (GFP) of Victoria aequoria (Chalfie, et al. (1994) Science 263:802-805), modified GFPs (Heim, et al. (1995) Nature 373:663-4; WO 96/23810), and the gene products encoded by the Photorhabdus luminescens lux operon (luxABCDE) (Francis, et al. (2000) Infect. Immun. 68(6):3594-600). Transcription and translation of these type of reporters leads to the accumulation of the fluorescent or bioluminescent proteins in test cells, which may be measured by a device, such as a fluorimeter, flow cytometer, or luminometer. Methods for performing assays on fluorescent materials are well-known in the art (e.g., Lackowicz (1983) In: Principles of Fluorescence Spectroscopy, New York, Plenum Press).

For convenience and efficiency, enzymatic reporters and light-emitting reporters are preferred for the screening assays of the invention. Accordingly, the invention encompasses histochemical, colorimetric and fluorometric assays.

A promoter which is operably associated or operably linked to nucleic acid sequences encoding a reporter means that the sequences are joined and positioned in such a way as to permit transcription. Two or more sequences, such as a promoter and any other nucleic acid sequences are operably associated if transcription commencing in the promoter will produce an RNA transcript of the operably associated sequences. A promoter is defined as a fully functional sequence which regulates expression of a nucleic acid sequence of the invention, i.e., a promoter which has binding sites for transcription factors and minimal promoter sequences. A promoter may include sequences upstream of a coding region of SEQ ID NO:1-13.

Various portions of a promoter or gene sequence may be generated by PCR (e.g., by inverse PCR methods using primers based on the 5′ sequences of SEQ ID NO:1-13 and genomic DNA as a template) or other conventional cloning techniques using DNA from the genomic locus of a gene identified herein. For PCR amplification, primers may be synthesized corresponding to the 5′ and 3′ boundaries of the selected promoter or gene regions. Primers also may contain additional restriction enzyme recognition secuences to facilitate subcloning.

Introduction of the reporter gene construct into the test cells may be carried out by conventional techniques well-known to those skilled in the art, such as transfection, transformation, conjugation, and transduction.

In addition to conventional chemical methods of transformation, the reporter gene construct of the invention may be introduced into a test cell by physical means, such as by electroporation or microinjection. Electroporation allows transfer of the vector by high voltage electric impulse, which creates pores in the plasma membrane of the cell and is performed according to methods well-known in the art. Additionally, the reporter gene construct may be introduced into test cells by protoplast fusion, using methods well-known in the art. The reporter gene construct may be introduced into a test cell transiently, or more typically, the nucleic acids are stably integrated into the genome of the test cell or remain as stable episomes in the test cell.

The test cells which contain the nucleic acid sequences encoding the reporter and which express products of the nucleic acid sequences encoding the reporter may be identified by at least four general approaches; detecting DNA-DNA or DNA-RNA hybridization; observing the presence or absence of marker gene functions (e.g., resistance to antibiotics); assessing the level of transcription as measured by the expression of reporter mRNA transcripts in the host cell; and detecting the reporter gene product as measured by immunoassay or by its biological activity.

The steps involved in a reporter-based screening assay include, culturing a test cell which contains nucleic acid sequences encoding a reporter operably linked to a promoter of a nucleic acid sequence of the invention; adding at least one test agent to a point of application, such as a well, in the plate containing the test cell and incubating the plate for a time sufficient to allow the test agent to effect reporter accumulation; detecting reporter activity of the test cell contacted with the test agent, wherein reporter activity indicates expression of the reporter polypeptide in the test cell; and comparing reporter activity of the test cell which has been contacted with the test agent to that of the test cell not contacted with the test agent. An increase in reporter activity of the test cell contacting the test agent relative to the reporter activity of the test cell not contacting the test agent indicates that the test agent causes an increase in expression of a nucleic acid sequence of the invention in the test cell.

Another embodiment of the screening method of the invention is based on the detection of gene expression using RT-PCR and/or microarray analysis. In this embodiment, at least one test agent is added to a test cell located, for example, in a well of a microtiter plate, and the test cell and test agent are incubated for a time sufficient to allow the test agent to effect accumulation of a nucleic acid sequence of the invention. Subsequently, expression or activation of said nucleic acid sequence is determined by reverse-transcription (RT) alone or in combination with array hybridization as disclosed herein. An increase in expression of said nucleic acid sequence in the test cell contacting the test agent, relative to expression of the same in a test cell not contacting the test agent, indicates that the test agent is useful for modulating synaptic growth or plasticity.

Test cells used in accordance with a screening method of the invention may be cultured under standard conditions of temperature, incubation time, optical density, plating density and media composition corresponding to the nutritional and physiological requirements of the cells. However, conditions for maintenance and growth of the test cell may be different from those for assaying candidate agents in the screening methods of the invention. Modified culture conditions and media are used to facilitate detection of the expression of a reporter molecule. Any techniques known in the art may be applied to establish the optimal conditions.

Screening assays of the invention may be performed in any format that allows rapid preparation and processing of multiple reactions such as in, for example, multi-well plates of the 96-well variety. Stock solutions of the agents as well as assay components are prepared manually and all subsequent pipeting, diluting, mixing, washing, incubating, sample readout and data collecting is done using commercially available robotic pipeting equipment, automated work stations, and analytical instruments for detecting the signal generated by the assay.

In addition to the agent and a test cell, a variety of other reagents may be included in the screening assays. These include reagents like salts, neutral proteins, e.g., albumin, detergents, etc. Also, reagents that otherwise improve the efficiency of the assay, such as protease inhibitors, nuclease inhibitors, anti-microbial agents, and the like may be used.

Activators or repressors of expression of a nucleic acid sequence of the invention may include agents which directly bind to a region on the promoter as well as agents which interact with transcriptional factors that bind to the said promoter thereby specifically regulating gene expression.

Agents identified in the screening assays provided herein are useful in stimulating or increasing synaptic growth or plasticity. Thus, these agents, as well as isolated proteins of the invention, or fragments thereof, may be formulated into pharmaceutical compositions comprising an effective amount of a recombinant protein or agent to increase the expression, protein levels, or activity of the inventive nucleic acid sequences or proteins encoded thereby. Such pharmaceutical compositions may be prepared by methods and contain vehicles which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000.

By effective amount it is meant an amount of recombinant protein or agent which stimulates or increases synaptic growth or plasticity by 25%, 50%, 75%, or more compared to individuals not receiving treatment. Preferably, the agent or protein provides both therapeutic treatment and prophylactic or preventative measures, wherein the object is to prevent or slow down (lessen) an undesired physiological condition, disorder or disease or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay or slowing of condition, disorder, or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Conditions, diseases or disorders which may be treated with an agent of the invention include, but are not limited to, Alzheimer's disease, Huntington's disease, benign forgetfulness of memory, dementia, and other diseases associated with synaptic loss.

A pharmaceutical composition may be administered to a mammal to stimulate synaptic growth or plasticity in said mammal. A mammal refers to any animal classified as a mammal, including humans, domestic and farm animals, and zoo, sports and pet companion animals such as a household pet and other domesticated animal such as, but not limited to, cattle, sheep, ferrets, swine, horses, poultry, rabbits, goats, dogs, cats, and the like. Preferred companion animals are dogs and cats. Preferably, the mammal is human.

A pharmaceutical composition of the invention may be administered by any suitable means, including parenteral injection (such as intraperitoneal, subcutaneous, intracranial or intramuscular injection), orally, or by topical application (e.g., transdermal or via a mucosal surface).

For oral administration, a pharmaceutical composition useful in the invention may take the form of solutions, suspensions, tablets, pills, capsules, powders, granules, semisolids, sustained release formulations, elixirs, aerosols, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin and acacia. Additionally, lubricating agents such as magnesium stearate, sodium lauryl sulfate and talc are often very useful for generating tablets. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention may be combined with various sweetening agents, flavoring agents, coloring agents, emulsifying agents and/or suspending agents, as well as such diluents as water, ethanol, propylene glycol, glycerin and various like combinations thereof.

The choice of formulation depends on various factors such as the mode of drug administration (e.g., for oral administration, formulations in the form of tablets, pills or capsules are preferred) and the bioavailability of the drug substance. Pharmaceutical formulations have been developed especially for drugs that show poor bioavailability based upon the principle that bioavailability may be increased by increasing the surface area, i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The term parenteral as used herein refers to modes of administration, which include intravenous, intramuscular, intraperitoneal, intrasternal, subcutaneous, intramedullary and intraarticular injection and infusion. A pharmaceutical composition for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Aqueous solutions are especially suitable for intravenous, intramuscular, subcutaneous and intraperitoneal injection purposes. Sterile, aqueous media are readily obtainable by standard techniques well-known to those skilled in the art. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

The pharmaceutical compositions useful in the present invention may also contain adjuvants such as, but not limited to, preservatives, wetting agents, emulsifying agents, and dispersing agents. Prevention of the action of microorganisms can be ensured by the inclusion of various antibacterial and antifungal agents, such as for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin.

In some cases, in order to prolong the effect of the drugs, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide, polyglycolide, and polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations may be sterilized, for example, by filtration through a bacterial-retaining filter, or by incorporating sterilizing agents in the form of sterile solid compositions which may be dissolved or dispersed in sterile water or other sterile injectable medium just prior to use.

Administration by slow infusion is particularly useful when intrathecal or epidural routes are employed. A number of implantable or body-mountable pumps useful in delivering compound at a regulated rate are known in the art. See, e.g., U.S. Pat. No. 4,619,652.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

For purposes of transdermal (e.g., topical) administration, dilute sterile, aqueous or partially aqueous solutions (usually in about 0.1% to 5% concentration), otherwise similar to the above parenteral solutions, are prepared.

The pharmaceutical compositions useful in the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In nonpressurized powder compositions, the active ingredients in finely divided form may be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

The compositions useful in the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see, e.g., Prescott (1976) Meth. Cell Biol. 14:33).

Other pharmaceutically acceptable carrier includes, but is not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Pharmaceutical compositions useful in the present invention may contain 0.1%-95% of an agent(s) of the invention, preferably 1%-70%. In any event, the composition or formulation to be administered will contain a quantity of a agent(s) according to the invention in an amount effective to treat the condition, disorder or disease of the subject being treated.

One of ordinary skill in the art will appreciate that pharmaceutically effective amounts of the agent may be determined empirically and may be employed in pure form or, where such forms exist, in pharmaceutically acceptable salt, ester or prodrug form. The agents may be administered to a patient as pharmaceutical compositions in combination with one or more pharmaceutically acceptable excipients. It will be understood that, when administered to, for example, a human patient, the total daily usage of the agents or composition of the present invention will be decided within the scope of sound medical judgement by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

For example, satisfactory results are obtained by oral administration of agents at dosages on the order of from 0.05 to 500 mg/kg/day, preferably 0.1 to 100 mg/kg/day, more preferably 1 to 50 mg/kg/day, administered once or, in divided doses, 2 to 4 times per day. On administration parenterally, for example, by i.v. bolus, drip or infusion, dosages on the order of from 0.01 to 1000 mg/kg/day, preferably 0.05 to 500 mg/kg/day, and more preferably 0.1 to 100 mg/kg/day, may be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging may also be arranged in a patient specific manner to provide a predetermined concentration of the agents in the blood, as determined by techniques accepted and routine in the art (HPLC is preferred). Thus patient dosaging may be adjusted to achieve regular on-going blood levels, as measured by HPLC, on the order of from 50 to 5000 ng/ml, preferably 100 to 2500 ng/ml.

The invention is described in greater detail by the following non-limiting examples.

### EXAMPLE 1

### Cell Culture Preparation

Time-mated pregnant rats were sacrificed by CO₂ asphyxiation in accordance with institutional guidelines for care and use of animals. Fetuses were removed by caesarean section and transferred to a sterile petri dish with phosphate-buffered saline. Fetal hippocampi were dissected from surrounding brain tissue and meninges were completely removed. Low-density cultures of dissociated embryonic day 18 rat hippocampi (Sprague Dawley from Hilltop Laboratories, Scottsdale, Pa.) were prepared using standard methods (Thakker-Varia et al. (2001) supra). Briefly, pooled tissue from each litter was mechanically dissociated in nutrient medium containing 7.5% fetal bovine serum and plated on poly-D-lysine-coated culture dishes at 350,000 cells/dish. Cultures were maintained in serum-free medium (Thakker-Varia et al., 2001) for 10-14 days and contained pure neurons.

### EXAMPLE 2

### Electrophysiological Recordings

Whole-cell patch clamp recordings were performed after 10-14 days in culture. Currents were recorded with an Axoclamp 200 amplifier, digitized at 2.5 kHz with an INDEC IDA 15125 interface, filtered at 5 kHz and stored. Recording parameters and stimulus protocols were controlled by custom software written with Borland C++ that utilizes device driver libraries supplied by INDEC. Data analysis programs were written with Microsoft Visual Basic. The. external bath solution for voltage clamp recordings was (in mM) 1.67 CaCl2, 1 MgCl2, 5.36 KCl, 137 NaCl, 17 glucose, 10 HEPES and 20 sucrose (neuron recording solution, NRS). The pipette solution contained (in mM) 105 Cs-methanesulfonate, 17.5 CsCl, 10 HEPES, 0.2 EGTA, 8 NaCl, 2 Mg-ATP, 2 Na-ATP, 0.3 Na-GTP, 20 phosphocreatinine, and 50 U/ml creatinine phosphokinase. All recordings were made at room temperature. The typical range of pipette resistance was 3-5 MΩ. Cell capacitance was 10-20 pF and access resistance was 7-20 MΩ. Vacuum perfusion system contained BDNF (50 ng/ml, Preprotech, Princeton, N.J.), NRS as a control or VGF peptides (0.1 μM=740 ng/ml). Three synthetic rat VGF peptides were used: Leu-Glu-Gly-Ser-28 (SEQ ID NO:15) amino acids 285-312 (amidated), Thr-Leu-Gln-Pro-62 (SEQ ID NO:14) amino acids 556-617 and Ala-Gln-Glu-Glu-30 (SEQ ID NO:16) amino acids 588-617. Each data point represents a cell from a separate dish and at least three different platings were used for each condition.

### EXAMPLE 3

### Data Analysis

Data were analyzed by integrating the synaptic currents for each sweep (synaptic charge). The charge measurements for all sweeps in a 1 min period were averaged (binned). Baseline is considered the average synaptic charge during the 2 min period (−2 to 0 min) in NRS immediately prior to BDNF or VGF application. Fold increases were then determined by dividing the synaptic charge during BDNF or VGF exposure by the baseline. Recordings were rejected if either the 0-5 min binned time period or 5-10 min binned time period after switching during BDNF or VGF exposure was 2×SEM below baseline, indicating “run down”.

### EXAMPLE 4

### Microarray

Total cellular RNA was prepared from neuronal cultures treated with either BDNF (50 ng/ml) or vehicle (water) for either 20 min or 3 hr by the guanidine isothiocyanate method and CsCl gradients (Thakker-Varia et al., 2001). DNAse-treated total RNA was prepared from neurotrophin or control hippocampal cultures and reverse transcribed using gene specific primers. Radioactively labeled probes were then hybridized to Atlas Arrays Rat 1.2 (Clontech, Palo Alto, Calif.) according to manufacturer's protocol. Arrays were performed in duplicate with an independent source of RNA. The hybridization pattern was analyzed on a phosphorimager and normalized to the global hybridization signal. Analysis was performed by Clontech using 1.7-fold change as criteria for significance. All transcriptional changes were confirmed by real time RT-PCR on independent samples (see below).

### EXAMPLE 5

### Real Time RT-PCR

cDNA (100 μl) was prepared from 2 μg control or BDNF-treated RNA using random primers and Superscript II reverse transcriptase. 25 μl PCR reactions were then carried out using gene specific primers designed by Primer Express software and TaqMan MGB probes (Applied Biosystems 7000 Sequence Detection System, Foster City, Calif.). Duplicate wells were included for each condition and primer pair. Primers specific to the housekeeping gene, glyceraldehyde-3-phosphate dehydrogenase (GAPDH) were used as an internal control. Data analysis was performed according to the protocol provided by Applied Biosystems.

### EXAMPLE 6

### Single-Sell aRNA Amplification

Following whole-cell patch clamp recording for approximately 30 min, the cellular contents were aspirated into the patch pipette with a small amount of suction. cDNA synthesis was performed in vitro using the MessageAmp aRNA kit (Ambion, Austin, Tex.). Two rounds of aRNA synthesis were performed resulting in a one million-fold amplification of the original material (Phillips and Eberwine, 1996). 20 μl of cDNA was synthesized from all of the aRNA and 2 μl was used as a template in real-time PCR for Arc and GAPDH gene expression as described above with the exception that 65 amplification cycles were used.

### EXAMPLE 7

### Pharmacological Treatments

Hippocampal neurons were pre-incubated with inhibitors for 30 min prior to BDNF treatment (20 min for IEGs and 3 hr for VGF). All inhibitors were purchased from Calbiochem, San Diego, Calif. K252a (200 nM), U0126 (30 μM), PD98059 (50 μM), U73122 (10 μM), KN93 (10 μM), LY294002 (30 μM), H-89 (1 μM) and chelerythrine chloride (CC, 1 μM). Pharmacological inhibitors were tested for efficacy by assaying downregulation of endogenous kinase phosphorylation in cell culture using Western blot (Y. Z. Du and C. F. Dreyfus, personal communication). Each experiment consisted of 4 groups: vehicle, inhibitor alone, BDNF, and BDNF plus inhibitor. Following inhibitor and BDNF treatments, RNA was isolated and subjected to real time RT-PCR as described above.

### EXAMPLE 8

### SDS-PAGE and Western Blot Analysis

BDNF-treated and untreated hippocampal neurons plated in equal numbers were solubilized in lysis buffer (20 mM Tris pH 8, 0.5% Triton X-100, 0.5% SDS, protease inhibitor tablet, 1 mM PMSF, and 0.5 mM vanadate). The protein content was determined with the BCA protein assay kit (Pierce Chemical Co, Rockford, Ill.). Samples containing equal amounts of protein were denatured in Laemmli's sample buffer (containing β-mercaptoethanol) for 5 min and subjected to 8- 12% gradient SDS-PAGE (Suen et al., 1997). The proteins were transferred to polyvinylidene difluoride (PVDF) membranes (Millipore Corp, Bedford, Mass.) which were blocked for 1 hour with a 5% solution of dry milk powder plus 5% normal serum in 0.1% TWEEN 20-PBS. The PVDF membranes were incubated with goat anti-R15 VGF (1:500) (Santa Cruz Biotechnology, Inc, Santa Cruz, Calif.) or goat anti-actin (1:500) (Santa Cruz) with 1% bovine serum albumin overnight at 4° C. Membranes were washed, followed by 1 h incubation with donkey anti-goat HRP-conjugated IgG (1:5000) at RT. The immunopositive bands were visualized by chemiluminescence using the ECL™ detection kit (NEN, Boston, Mass.), quantitated on a BIO-RAD Gel Doc and normalized to actin.

### EXAMPLE 9

### Immunocytochemistry

Cultures were fixed in 4% paraformaldehyde. A blocking step was carried out in 30% normal horse serum in PBS containing 0.3% Triton X-100. VGF antibody (R15) (1:100) (Santa Cruz) was applied overnight at 4oC. Cultures were incubated for 1 hr at room temperature with Alexa Flour 594 donkey anti-goat secondary (1:500) (Molecular Probe, Eugene, Oreg.) and visualized on a Leica fluorescent microscope (DMIRB).

### EXAMPLE 10

### Trace Eyeblink Conditioning

Classical eyeblink conditioning methods followed published procedures (Donahue et al., 2002; Leuner et al., 2003). Generally, an auditory stimulus is associated with stimulation to the eyelid. After repeated pairings, the animals respond to the auditory stimulus in anticipation of the eyelid stimulation. When a temporal gap is placed between the auditory stimulus and the eyelid stimulation, learning becomes hippocampal-dependent (Beylin et al., 2001). Specifically, groups of rats were exposed to 200 trials per day for 4 days for a total of 800 trials. The rats were trained with a trace paradigm in which a 250-ms 82-dB burst of white noise (CS) was separated from a 100-ms, 0.7-mA periorbital shock (US) by a 500-ms trace interval. The intertrial interval was 25±5 s. During unpaired training, rats received the same number of CS and US exposures, but presented in an explicitly unpaired manner. Naïve rats did not receive stimulus exposure. Rats were anesthetized with Nembutal (50 mg/kg) and decapitated immediately after training. The hippocampus was dissected and frozen on dry ice. RNA was prepared from the tissue and subjected to real time RT-PCR as described above.

1. A method for modulating synaptic growth or plasticity comprising increasing the expression of a BNDF-inducible nucleic acid sequence or activity of a protein encoded thereby, so that synaptic growth or plasticity is stimulated.

2. The method of claim 1 wherein the nucleic acid sequence which is inducible by BDNF comprises c-fos proto-oncogene (SEQ ID NO:1); early growth response protein 1 (SEQ ID NO:2); activity-regulated cytoskeletal associated (SEQ ID NO:3); fos-related antigen 2 (SEQ ID NO:4); G1/S-specific cyclin D1 (SEQ ID NO:5); voltage-gated potassium channel protein (SEQ ID NO:6); sodium channel, beta 1 subunit (SEQ ID NO:7); secretogranin II precursor (SEQ ID NO:8); somatostatin receptor 4 (SEQ ID NO:9); transmembrane receptor UNC5 homology (SEQ ID NO:10); neuropeptide Y (SEQ ID NO:11); VGF protein precursor (SEQ ID NO:12); or protein-tyrosine phosphatase 1B (SEQ ID NO:13).

3. A method for identifying an agent which increases synaptic growth or plasticity comprising contacting a test cell with an agent and detecting activation of:
c-fos proto-oncogene (SEQ ID NO:1);
early growth response protein 1 (SEQ ID NO:2);
activity-regulated cytoskeletal associated (SEQ ID NO:3);
fos-related antigen 2 (SEQ ID NO:4);
G1/S-specific cyclin D1 (SEQ ID NO:5);
voltage-gated potassium channel protein (SEQ ID NO:6);
sodium channel, beta 1 subunit (SEQ ID NO:7);
secretogranin II precursor (SEQ ID NO:8);
somatostatin receptor 4 (SEQ ID NO:9);
transmembrane receptor UNC5 homology (SEQ ID NO:10);
neuropeptide Y (SEQ ID NO:11);
VGF protein precursor (SEQ ID NO:12); or
protein-tyrosine phosphatase 1B (SEQ ID NO:13) nucleic acid sequences in the test cell wherein an increase in the activation of said nucleic acid sequences in the test cell contacted with the agent relative to the activation of said nucleic acid sequences in a test cell not contacted with the agent is indicative that said agent increases synaptic growth or plasticity.

4. A method for treating a disease or condition associated with damaged or diseased synapses comprising administering an effective amount of an agent identified by the method of claim 3.


SEQUENCE LISTING (16 sequences)

SEQ ID NO: 1 - DNA, 2116 bp, Rattus rattus
cgcaactgagaagactggatagagccggcggagccgcgaacgagcagtgaccgcgctcccacccagctctgctctgcagctcccaccagtgtctacccctggacccctcgccgagctttgcccaaaccacgaccatgatgttctcgggtttcaacgcggactacgaggcgtcatcctcccgctgcagtagcgcctccccggccggggacagcctttcctactaccattccccagccgactccttctccagcatgggctcccctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccaccccgtcgaccggggcttacgccagagcgggagtggtgaagaccatgtcaggcggcagagcgcagagcatcggcagaaggggcaaagtagagcagctatctcctgaagaggaagagaaacggagaatccgaagggaaaggaataagatggctgcagccaagtgccggaatcggaggagggagctgacagatacgctccaagcggagacagatcaacttgaagacgagaagtctgcgttgcagaccgagattgccaatctactgaaagagaaggaaaaactggagtttattttggcagcccaccgacctgcctgcaagatccccaatgacctgggcttcccagaggagatgtctgtgacctccctggacttgactgggggtctgcctgaggctaccaccccagagtctgaggaggccttcaccctgcctcttctcaatgaccctgagcccaagccatccttggagccggtcaagaacattagcaacatggagctgaaggctgaaccctttgatgacttcttgtttccggcatcatctaggcccagtggctcggagactgcccgctctgtgccagatgtggacctgtctggttccttctatgcagcagactgggagcctctgcacagcagttccctggggatggggcccatggtcacagagctggagcccctgtgcactcccgttgtcacctgcactcccagctgcactacctatacgtcttcctttgtcttcacctaccccgaggctgactccttccctagctgcgcagctgcccaccgaaagggcagcagcagcaacgagccctcctctgactcactgagctcgcccacactgctagccctgtgagcagtcagagaaggcagggcagccggcactgactgagctggtgcattacagagaggagaaacacgtcttccctcgaggggttcccgtagacctagggaggaccttatctgtgcgtgaaacacaccaagctgtggacctcaaggacttgaaagcatccacatctggactccagtcctcacctcttccggagatgtagcaaaaaaacaaaaaaacaaaacaaaaaaaaaacaaaacaaaaaatcaaaagcaaccgcatggagtgtattgtttgtagtgacacctgagagctggtagttagtagcatgtgagccaggcctgggtctgtgtctcttttctctttctccttagtcttctcatagcattaactaatctgttgggttcattattggaattaacctggtgctggatatttttcggattgtatctagtgcagctgattttaacaatacctactgtgttcctggcaatagtgtgttccaatttagaaatgaccaatattaaactaagaaaagatagaactttattttccggtagatagaaataaatcgctatatccacgtactgtagctcttcagcgtccatgttcattgtcatgtaactgatcatgcattgttgaggtggtctgaatgttctgacattaacagttttccatgaaaacgttttattgtgttttcaatttatttattaagatggattctcagatatttatatttttattttatttttttctatcctgaggtctttcgacatgtggaaagtgaatttgaatgaaaaaattttaagcattgtttgcttattgttccaagacattgtcaataaaagcatttaagttgaatgcg

SEQ ID NO: 2 - DNA, 3112 bp, Rattus norvegicus
cgcagaacttggggagccgccgccgcgattcgccgccgccgccagcttccgccgccgcaagatcggcccctgccccagcctccgcggcagccctgcgtccaccacgggccgcggccaccgccagcctgggggcccacctacactccccgcagtgtgcccctgcaccccgcatgtaacccggccaacatccggcgagtgtgccctcagtagcttcggccccgggctgcgcccaccacccaacatcagctctccagctcgcacgtccgggatggcagcggccaaggccgagatgcaattgatgtctccgctgcagatctctgacccgttcggctcctttcctcactcacccaccatggacaactaccccaaactggaggagatgatgctgctgagcaacggggctccccagttcctcggtgctgccggaaccccagagggcagcggcggcaataacagcagcagcagcagcagcagcagcagcgggggcggtggtgggggcggcagcaacagcggcagcagcgctttcaatcctcaaggggagccgagcgaacaaccctacgagcacctgaccacagagtccttttctgacatcgctctgaataacgagaaggcgctggtggagacaagttatcccagccaaactacccggttgcctcccatcacctatactggccgcttctccctggagcctgcacccaacagtggcaacactttgtggcctgaaccccttttcagcctagtcagtggccttgtgagcatgaccaaccctccaacctcttcatcctcagcgccttctccagctgcttcatcgtcttcctctgcctcccagagcccacccctgagctgtgccgtgccgtccaacgacagcagtcccatttactcagctgcacccacctttcctactcccaacactgacatttttcctgagccccaaagccaggcctttcctggctctgcaggcacagccttgcagtacccgcctcctgcctaccctgccaccaagggtggtttccaggttcccatgatccctgactatctgtttccacaacaacagggagacctgagcctgggcaccccagaccagaagcccttccagggtctggagaaccgtacccagcagccttcgctcactccactatccactatcaaagccttcgccactcagtcgggctcccaggacttaaaggctcttaataacacctaccagtcccaactcatcaaacccagccgcatgcgcaagtaccccaaccggcccagcaagacacccccccatgaacgcccgtatgcttgccctgttgagtcctgcgatcgccgcttttctcgctcggatgagcttacacgccacatccgcatccatacaggccagaagcccttccagtgtcgaatctgcatgcgtaatttcagtcgtagtgaccaccttaccacccacatccgcacccacacaggcgagaagccttttgcctgtgacatttgtgggagaaagtttgccaggagtgatgaacgcaagaggcataccaaaatccacttaagacagaaggacaagaaagcagacaaaagtgtcgtggcctcctcagctgcctcttccctctcttcctacccatccccagtggctacctcctacccatcccccgccaccacctcatttccatccccagtgcccacctcttactcctctccgggctcctctacctacccgtctcctgcacacagtggcttcccatcgccctcggtggccaccacctatgcctccgtcccacctgctttccctgcccaggtcagcaccttccagtctgcaggggtcagcaactccttcagcacctcaacgggtctttcagacatgacagcaaccttttctcctaggacaattgaaatttgctaaagggaatgaaagagagcaaagggaggggagcgcgagagacaataaaggacaggagggaagaaatggcccgcaagaggggctgcctcttaggtcagatggaagatctcagagccaagtccttctagtcagtagaaggcccgttggccaccagccctttcacttagcgtccctgccctccccagtcccggtccttttgacttcagctgcctgaaacagccacgtccaagttcttcacctctatccaaaggacttgatttgcatggtattggataaaccatttcagcatcatctccaccacatgcctggcccttgctcccttcagcactagaacatcaagttggctgaaaaaaaaaatgggtctgggccctcagaaccctgccctgtatctttgtacagcatctgtgccatggattttgttttccttggggtattcttgatgtgaagataatttgcatactctattgtactatttggagttaaattctcactttgggggagggggagcaaagccaagcaaaccaatggtgatcctctattttgtgatgatcctgctgtgacattaggtttgaaacttttttttttttttgaagcagcagtcctaggtattaactggagcatgtgtcagagtgttgttccgttaattttgtaaatactgctcgactgtaactctcacatgtgacaaaatacggtttgtttggttgggttttttgttgtttttgaaaaaaaaattttttttttgcccgtccctttggtttcaaaagtttcacgtcttggtgcctttgtgtgacacaccttgccgatggctggacatgtgcaatcgtgaggggacacgctcacctctagccttaagggggtaggagtgatgtttcaggggaggctttagagcacgatgaggaagagggctgagctgagctttggttctccagaatgtaagaagaaaaatttaaaacaaaaatctgaactctcaaaagtctatttttttaactgaaaatgtagatttatccatgttcgggagttggaatgctgcggttacctactgagtaggcggtgacttttgtatgctatgaacatgaagttcattattttgtggttttattttacttcgtacttgtgtttgcttaaacaaagtgacttgtttggcttataaacacattgaatgcgctttactgcccatgggatatgtggtgtgtatccttcagaaaaattaaaaggaaaataaagaaactaactggt

SEQ ID NO: 3 - DNA, 3032 bp, Rattus norvegicus
agtgctctggcgagtagtcctccctcagccgcagtctctgggcctcttcagcttgagcggcggcgagcctgccacactcgctaagctcctccggcaccgcgcacttgccactgccactgccgcttcgcgcccgctgcagccgccggctctgaatccttctggcttccgcctcagaggagttcttagcctgtcccgaaccgtaaccccggcgagcagatggagctggaccatatgacgaccggcggcctccacgcctaccctgccccgcggggtgggccggccgccaaacccaatgtgatcctgcagattggtaagtgccgagctgagatgctggagcacgtacggaggacccaccggcatctgttgaccgaagtgtccaagcaggtggagcgagagctgaaagggttgcacaggtcggtgggcaagctggagaacaacttggacggctatgtgcccacgggcgactcacagcgctggaagaagtccatcaaggcctgtctctgccgctgccaggagaccatcgccaacctggagcgctgggtcaagcgtgagatgcacgtgtggagggaggtcttctaccgtctggagaggtgggccgaccgcctggagtccatgggcggcaagtacccagtgggcagcgagccggcccgccacactgtctctgtaggtgtggggggtccagagccctactgccaggaagctgatggctacgactacactgttagcccctatgccatcaccccgccacctgccgcaggagagctgcctgagcaggagtcagttggggctcagcaataccagtcttgggtgccaggtgaggatgggcaaccaagcccaggtctggatacccagatctttgaggacccacgggagttcctgagccacctggaagagtacctgcggcaggtgggtggctctgaagaatattggctgtcccagatccagaaccacatgaatgggccagccaagaagtggtgggagttcaaacagggctcggtgaagaactgggtggagttcaagaaggagtttctgcagtacagtgagggtacgctctcccgcgaagccattcagcgggagctggacctgccacagaagcagggtgagccacttgaccagttcctctggcgtaagcgggacctgtaccagacactgtatgtggacgctgaggaggaggagatcattcagtatgtggtgggcaccctgcagcccaagttcaagcgctttctgcgccacccacttcccaagaccctggagcagctcatccagaggggcatggaagttcaggacggcctggagcaggcagctgagccttctgtcacccctctgcccacagaggatgagactgaggcactcacgcctgctcttaccagcgagtcagtagccagtgacaggacccagcctgaatagaggggccagcccagggtccccagcctgcctgccacacccagtctgtggcttttgtcaactaggacttgattgagctggggctgacacccaaggggatgccctgtccagccagacaccttctcacccactggcctgactcacaactgccacacaaccatgattcatggacatcaagaagcccctctcccatagggctcccacctgccacctacccctcacctgtctgccctagtcctggccctgtctccagtggcctcaccctctacactctcagaccatcacagaacacctttggcttcctcattctgcatcagtgtccagggccctttgggtagtcaagaaatcaagtgtctgaaaggcaatgaaaagtaggcaccaaacccaaggggcatcccagggcagatgctaaagcagaatcagagatggccgaaggaacctctacttccggggatgcagcccgctcctacagacacagcagatccagctggtgccctacctgcctcccagagcaactggccagtcttgggcagcatagctcccctctcagggtgagctgaagcagcagacctgacgcgctggcgcctcctggcccccagcagtgattcataccagtgaagaaaagcagacttcggctccatgactcagccatgccaggcggagggtcccagaggggctgagtcctcagccccagctgaggcagcagctggagtcttcagagccaggtgaatgacaccaggtctcaagctgctgagaagtctttccggccatgtctggaaggggtaccaccccagcaccagcaccgtcccctcctctcttgaagctgcctgcacagaggttccaagacactttcaaggcagagaaaataggattacaaagaggaggtgccttggcagagggcagcacccagctcagcctcagagctgaaggtgaagacaagccagcgtgaaaccccgggtctgccacgaatgcccgctccgctggccactcaccagctgcctgccacaagccactgcagcttgagcagggtctgtgccctctcagcacagagcccagttcgctgcgtggcctttggcccccgccagaaccttgcaggagccttaaggttcgggccctagcccagcctgaccttacctgctgtgccctgcctgctggtcaagtccagtcccaggagaccccatgccttggctcctaggctgttccaggcacttccctgacctgccgggtgattgcccagctggaacctcatccacaccccagcaccaaccacctcgtgttggtaactgctcgtgtctgtagtctgagtaggccatgttgaggttcctccatctgcctggtccattggtgttctgagaccagttccactgctgttctgacagatcccccaccctgtgcccctgccagcccccacaggtttatttttgcacataaaccatgacccatactaatttggctagctctggggactagggagaccctggagatctcaagagtgtggctatcccctattttcaccaagccttcaatatccagccaggccatctggcccacaccatcttacctcaaagacagacatatatatatatatacatatatatgattttgttaataaaactatgaaatttaaa

SEQ ID NO: 4 - DNA, 984 bp, Rattus norvegicus
atgtaccaggattatcccgggaactttgacacctcgtcccggggcagcagtggctctcctgcgcacgccgagtcctactccagcggtggcggcggccagcagaagttccgggtagatatgcctggctcaggcagtgccttcatccccacaatcaacgccatcaccaccaccagccaggacctgcagtggatggtacagcccacagtgattacctccatgtccaacccctatccgcgctcgcacccctacagccccctgccaggcttgcgatcagtccctgggcacatggctctccccagaccaggagtgatcaagaccattggtaccaccgtgggccgcagaaggagagatgagcagctgtctcccgaagaggaggagaagcgtcgaatccggcgagagagaaacaagctggccgctgccaagtgtcggaaccgtcgacgtgagctgacggagaagctgcagacggagacagaggagctggaggaggagaagtccggtctgcagaaagaaatcgctgagctgcagaaggagaaggagaagctggagttcatgctggtggctcacggccctgtgtgcaaaatcagtcctgaggaacgccgatcttcccccacctccggggtacagtccttacgcggtacgggcagtgctgtcggccctgtggtggtgaaacaggagcctcctgaagaggacagcccctcttcctcagcagggatggacaagacccagcgctctgtcatcaagcccattagcattgctgggggtggtttctacggggaagagcctctgcacacccccatcgtggtgacctccacacctgccatcactccgggcacttcaaaccttgtcttcacctaccccagtgtcctggagcaggagtcgcctgcgtcgccctccgagtcctgttccaaggctcaccgcagaagcagtagcagtggggaccagtcatcagactccttgaactcccccactctgctggctctgtaa

SEQ ID NO: 5 - DNA, 3751 bp, Rattus norvegicus
agaaagagagctacagactccgcgcgctccggaagaccgggtcgtagagctcgagacaactcccgccagcagcaagacacgggagacccaaccaggatgacaaccctccccaggccgcccgctccatggaacaccagctcctgtgctgcgaagtggagaccatccgccgcgcgtaccctgacaccaatctcctcaaccgaccgggtctgcgagccatgcttaagactgaggagacctgcgcgccctccgtttcttacttcaagtgcgtgcagagggagattgtgccatccatgcggaaaatcgtggccacctggatgctagaggtctgcgaggagcagaagtgcgaagaggaggtcttcccgctggccatgaactacctggaccgtttcttgtctctggagcccctgaagaagagccgcctgcagcttctgggggccacctgcatgttcgtggcctctaagatgaaggagaccattcccctgactgccgagaagttgtgcatctacactgacaactctatccgccccgaggagttgctgcaaatggaactgcttctggtgaacaagctcaaatggaacctggccgccatgactccccacgatttcatcgaacacttcctctccaaaatgccagaggcggatgagaacaagcagatcatccgcaaacatgcacagacctttgtggccctctgtgccacagatgtgaagttcatttccaacccaccctccatggtggctgctgggagtgtggtggccgcgatgcagggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacatcgatcccaaggccaccgaggaggaaggggaagtggaggaagaagctggtctggcttgcacacccaccgacgtccgagatgtggacatctgagggcccaccgggcaggcgggagtcaccaagtagtggcatccgcgaagagaaaggagccagcccgggtgctcctgacgaggtcccccttggggatgtgttgttaccagaaggggaagttttgttctctttgttggttgtttttccttaatctttctcctttctatctgatttaagcaaaagagaaaaaaatacctgaaagctgtcttaaagagagagagagagatagaattcgcatcaccctgagtatagggagacgggggggtgcctacaaaaatagaattctgtaccccagtaatcaactagttttctattaatgtgcttgtctgttctaagaataggattaacacacaggaagtcttgagaaggatttttgattcttttatgtgtttaagaaaaaaaagcttaagaaacattgctttaaaaaaagaaggaaaaaaaatacagcaaaccattgttaaagtagaagggttttaggtagagaaatgtactctgctttggtgaaaaggcacagatttgggaaggtcctcaacctcactcccctgcttgttcagtgcctacagacctgttacctgatgatacctgtgctttatcccaggagtggggcagacctcttaaccttactgatggtcgattcgacctctagcgggctcgtgggtgtggcaacctccagggctcatgttatgggccctctgccccccacaacctgcaggttcacagagcgccagccagcacgcaggggtagggatgaaatagtgacataatatattctatttttggggaaccttcctgttttgggggttcctgtttagagagatgctggtttttggctggtggccctgcagcccactcaacatcaggttgaacccacagcttttcttttgtgtgtggtttgttttgttatgtttttccttctccatgttcccaaaaccattccatttcaaagcacttttggtcagctagctggaggcagtgttgctgggtgtgtgtgtgtgtgtgtgtgtgtgtgtgtgtgtgtgtgtgtgtcggtgggggagggggtctagtggaatggttggggatgtccacacactcaattctgatggctgcacagcaggtgtgtagggctggtagcatgaggtgcttgggaagttgtgttgggtcgagaagagaaagctctgttctcgtaccaccgggatcttcctgcaaagtcggagggatcttttggtgccagctggcgtttggaagtagggaccgtgatggcattacctggacaaggggattcaggacgactcttaagtctttcacacaggaggcttttaaacactaaaatctctaatttatacgtaaggctacagaagagtatttattgggaaggttgcccatgacccgtgtgactcaaagcaatgtgatcttcccttgattcaaacgcacaccttcttccctgctggcgaaggttttaggccattgtctgagagactggtcttttattggccaagtggggtgtcctccaaaaaaaccaaaccacaaagacggagatgtgggtctccttgactttcccaacccaattgcccccattggagagccatccaaacctgaggaaaattaggggaccccaaaagagtcttgagtctggcacattcttgccgccgctcccaagttagcaacagtaggtaatttgcacacctctggctctgtgcctttctgttcaggatgtgttggtgggaggtggagagcgggtggctggagaggggatgtgagagaagaagtatggagggtagggaccacacgggacagaccgcggctcctttcacagcgctgctaccaatgactcccaggatccccggcgttcggaaccagattcacgttgctttgtatctttcatgttgttttcgctgctattggagggtcagttttattttgttgttgttggtttgtttgtttttttttacaatgtcataaactgccatgttcaagttttaatttcctcgtagaagagtgtatttacagatgccctctccgggactttttctatttattgtgattcaattttggattaatgtgattaccgctgtattcccaacaaaaggtgttttccacacacacacacacacacacacacacacacacacacacacacaggttcctgtacacaatacctcatgtatcacctaacaatacataggcctgccaggcgggtggggcggtctgcctccagggaccctgggaccctgctggggatcatcctgtcatgctgggccttcatttgatctgggacatagcatcacagcagtcaggccaactgtgttctgttagttatcaatattgttacttgtagcggcctgttgtgcatgccaccatgctgctggacccggagagatttgttctgagtctctggtgcatcatataatctgttaggttctagtgttctgtcttgttttgtgttactcacagcattgtgctaatgtaaagccagccgcaatgctgtaggccccaggttccctagcaagctgccaaaccaaaagggtcaccaccagcttagctgaggcgtcccaaccaggcaggacccttgagggctgctgtgtccatggtgatggggtgaagttttggccaaagggccaaaggctggtggatccacacagtctgccctgtgacatgaatggctttgaggggctctggctggtggtcaggttggctttgtgtattctggttgacacaccatggcgcttcccagcacagacatgtgaccagcatggtccagg

SEQ ID NO: 6 - DNA, 1746 bp, Rattus norvegicus
tcctggcctcctacccctgcaccctgcatccatcatgacggtgatgtcaggggagaatgcagacgaggcttcggccgctccaggtcacccccaggatggcagctacccaaggcaggcggaccacgacgaccacgaatgctgcgagcgcgtggtgatcaacatctccgggctgcgcttcgagacgcagctcaagactctggcccagttccccaacacgctgctgggcaacccgaagaaacgcatgcgctactttgaccctctgaggaatgagtacttctttgaccgcaaccggcccagcttcgatgccatcctttattactaccagtcgggggggcgcctgcgcaggccggtcaacgtgcccctggacatgttctccgaggagattaaattttacgagttgggcgaggaggccatggagaagttccgggaagatgagggcttcatcaaggaagaggagcgccccctacccgagaaggagtaccagcgccaggtgtggctgctctttgagtatccggagagctcaggacctgcacgggttattgccattgtatccgtcatggtcatcctcatctccatagtcatcttttgcctggagactctccctgagctgaaggatgacaaggacttcacgggcaccattcaccgcatcgataacaccacagtcatctacacttctaacatcttcacagaccctttcttcattgtggaaaccttgtgtatcatctggttctcttttgagctggtggtgcgcttcttcgcctgccccagcaagacagacttctttaagaacatcatgaacttcatcgacattgtggccatcatcccttatttcattaccctgggcacagagatagctgagcaggaggggaatcagaagggcgagcaggccacttccctggccatcctcagggtcatccgcttggtaagggtgttcagaatcttcaaactctcccgccactccaagggccttcagatcctgggccagaccctcaaagctagtatgagggagttagggctgctcatctttttcctcttcattggcgtcatactgttttctagtgcagtgtactttgcggaggcggaagaagctgagtcgcacttctccagtatccccgatgctttctggtgggcggtggtgtccatgaccactgtgggatacggtgacatgtaccctgtgacaattggaggcaagatcgtgggctccttgtgtgccatcgctggtgtgctgacaattgccctgcccgtacctgtcattgtgtccaatttcaactatttctaccaccgagaaactgagggggaagagcaggctcagttgctccatgttagttctcctaacttagcctctgacagtgacctcagccgccgcagctcctctactatcagcaagtctgagtacatggagatcgaagaggacatgaacaatagcatagcccactacaggcaggctaatatcagaactggtaactgcaccgcaactgatcaaaactgcgttaataagagcaagctcctgaccgatgtttaaaaaaagcaccaggcaagcaatcaaaagcccccaaacaaaacccttggcgactcctgtcccactctgtagatactttactaaaaccgtagtctttgaatgctttatttaactggcaatgcactgttgcattgtgaatttggggggtgggcaaacctgaagctttcaagatcacatttaaaaaacaaaaccaaccaaacaagcaaaaagaaaaaaaaaacccaaca

SEQ ID NO: 7 - DNA, 1490 bp, Rattus norvegicus
cgcagcctggatgcgccctgtggcgcacgcacgcagcatcccgagcctcccgccgcgcgcgggatgcctgctctccgggccccggggcttggccccggcggtaaccggagcgggggggcgcgcccccccagcagcagctgcggcgcccgcgcccgggccagtcgccgccggggcccatctcctgtcgccgcgctctgcgacccaccgccttgcgcggccatggggacgctgctggctctcgtggtgggcgcggtgctggtatcctcagcctgggggggctgcgtggaggtggattctgagaccgaggcagtgtatgggatgaccttcaaaatcctgtgtatctcctgtaagcgtcgtagtgagaccaccgccgagaccttcacggagtggaccttccgccagaagggcacagaggaatttgtcaagatcctacgctatgagaatgaggtgctgcagctggaggaagatgagcgctttgagggccgtgtggtgtggaacggtagtcggggcaccaaggacctgcaggacctgtccatcttcatcaccaatgtcacctacaaccactctggcgactacgaatgtcacgtctaccgtctcctcttctttgataattacgagcacaacaccagcgtcgtcaagaagatccacctggaggtggtggacaaggccaacagagatatggcatccatcgtgtcagagatcatgatgtacgtgctcattgtggtgttaaccatatggctcgtggcggagatggtgtactgctacaagaagattgctgctgccacggaagctgctgcacaagagaatgcctcggaatacctggccattacttccgagagcaaagagaactgtacaggcgtccaggtggctgaatagcgctggctctgggctccgcctcaaggaagagccagcctacgggtaccctccagccctgcagtggggatcagcccctggtgggtaccctcccctggcagtggggatcagcccatcggtctccccagcctcacagttctgcagtggagccaccagggtgggagcgggcagggactgatcccacctcacccaccgcctcccacctaccctcccaccgccatgcatgatgggtgaagcaatatggccgccccaccctgcttttgctgcctgtttgggggagggggcggtgaggcgagggggcaggccccgcccccttctttttgctgatttgcacataggccacttccacacgcactgccaggccagccggcccacccctgcttgatggggtgaagaggggtcgggacagggacagtagtgggcagggggttctgggcctcatctcccctccgcttcctccggctggacctggggttcccttcctgtgacacctcctagccctggcccacccgccctctctcaccagccttcaattgtggtctcttgggaaggcctcttcggcctcctatctttacagaagtagtttttgttcatgaaataaagattcttggactcga

SEQ ID NO: 8 - DNA, 2289 bp, Rattus norvegicus
acaatataagacagaggaaaattttaagacatgactgaatcgaaggcttaccgatttggagcagttctgcttcttatccacttaattttccttgtccctggaaccgaagcagcttccttccagcgaaaccagctgcttcagaaagaaccagacctcagattggagaatgtccagaagtttcctagtccagaaatgatcagggctttggagtacatagaaaagctcaggcagcaggcccacagagaagaaagcagcccagactacaatccctaccaaggcatctctgttccccttcaactcaaagaaaacggagaagaaagtcacttggcagagagctcaagggatgtcctgagtgaagacgagtggatgcggataatacttgaggctttgaggcaggctgaaaatgagccgccatctgccctcaaggagaacaagccctatgccttgaatctggagaagaacttccctgtggacacgcctgatgactatgagactcaacaatggcctgagaggaaactcaagcacatgcggttccctctcatgtatgaagagaattccagggaaaaccccttcaaacgcacaaacgaaatagtagaagaacagtacacaccccaaagtcttgctaccctggagtctgtgttccaagagcttgggaaactgacagggccaagcaaccagaagcgtgagagggttgacgaggaacagaagctctacacggacgatgaagatgacgtgtacaagaccaacaacattgcctatgaagatgtggtcgggggagaagactggagtcctatggaggagaaaatagagactcaaacccaggaagaggtgagagacagcaaagagaacacagaaaaaaacgaacaaatcaatgaagagatgaaacggtcagggcacttggggctcccagatgaaggtaaccggaaagagagcaaagaccagctctcagaggacgcctccaaggtcatcacctacttgagaaggttagtgaatgctgtgggcagtgggaggtcccagagtgggcaaaacggggacagggcagccaggcttcttgagaggccccttgattctcagtctatttatcagctgattgaaatctccaggaatttgcagataccccctgaagacttaattgagatgctcaaagctggggagaaaccaaatgggttggtggagcccgagcaggatctggagcttgctgttgacctagatgacatcccggaagctgacatagaccgcccagacatgtttcaaagtaagacgctctccaagggtgggtatcccaaggcacctggtcgaggtatgatggaggccttgccagatggcctcagtgttgaagacattttaaatgttttagggatggagaatgtagcaaatcagaagtccccatatttccccaaccaatacagccgagacaaggctctgctgaggcttccttatggtcctgggaaatctagagccaaccagattcccaaagtagcctggatcccagacgttgaaagcagacaagccccctatgacaatctgaatgataaggaccaagaattgggagagtacttagccaggatgctagttaagtaccctgagctcatgaataccaaccagctgaagagagtgcccagcccaggctcctcagaagatgacctccaagaagaagagcagctcgagcaggccatcaaggagcatctgggtcaaggaagctcccaggaaatggagaaactggccaaggtgagcaaaaggatccctgcaggatccctgaagaatgaggataccccaaatagacagtacctggatgaagatatgctcctgaaagtgctagagtatctcaatcaagaacaggcagagcagggaagggaacatcttgccaaacgggccatggaaaacatgtaaacagctttaatgcccaatttcccttcttttccccaagtgaatcccctccctttctcttaagtgtgttaatctctatcctgttaacactgtaatatctttaagtgatgtacaagcagatgactccagatagttttggggatctgctttacttattctgagctgttatgttgtgtatggatgtgtataaatgttatgactctcagattaaaaatatgtcctttattcaagaaagatatctatgatagtgttgactaatgtatccaatggtcatggtattgacaatgctcacatatgatgaagagtatcctataattatcttggaagtttttaacatttattgaattattttgttactgtctgtagtgttttgtggagttctggagcaaaatcaataaagca

SEQ ID NO: 9 - DNA, 1865 bp, Rattus norvegicus
gttcagcgttcggctgctctccacggcaatccgctgccccgggtgggcaccccgaagcatgaacacgcctgcaactctgcccctggggggcgaggacaccacctggacccctgggatcaacgccagctgggctccggatgaggaggaggatgcagtgcggtccgacggcacggggacagcgggcatggtaactatccagtgcatctatgcgctcgtgtgtctggtgggcctggtaggaaacgccctggtcatattcgtgatcctacgctatgccaaaatgaagacagccaccaacatctacctgctcaacctggccgtcgctgatgagctcttcatgctcagtgtgccatttgtggcctcggcggctgccctgcgccactggccgttcggggcggtgctgtgccgcgcagtgcttagtgtggacggccttaacatgttcacgagtgtcttctgcctcacagtgctcagcgtggatcgctatgtggctgtagtgcaccctctgcgagctgccacctaccggcggcccagcgtggccaagctaatcaacctgggagtgtggctagcatccttgctggtcaccctgcccatcgcagtcttcgctgacactaggccagctcgtgggggtgaggcagtagcttgcaacctgcactggcctcacccggcctggtctgcagtctttgtgatctatacttttttgctgggcttcctactcccggttctggctatcggattatgttacctgcttatcgtgggcaagatgcgtgctgtggccctgcgggctggctggcaacaacggaggcgctcagagaagaagatcactaggctcgtgctaatggtggtgactgtctttgtgctatgctggatgccattctatgtagtgcagcttctgaatctgtttgtcaccagcctcgatgccactgtcaaccatgtgtccctcatcctcagctatgccaacagctgtgccaacccgattctctatggtttcctctcagacaacttccgacgctctttccagcgggttctgtgcctgcgctgctgtctcctggaaacaactggaggtgctgaggaagagcccctggactactatgctactgctctcaaaagcagaggtggcccaggatgcatatgccctccattgccctgccagcaggagcccatgcaagcagaacctgcctgcaagcgagtccctttcaccaagaccactactttctgaaaaccatttcaccctccctcagcccacctgcaagcaggtctgcaccacactctcaagccagcaacttcaagaaaactcctgttgtcactaagccaggccctttcagcagcctgtgttctgtccctaggagcctcaggactcctgctagcccctgcctctcctaggactgactggctccaaggacaactccgtgggggtaggacttctctgggttttgggctagagtaccatccatcctttcctggacctctagcaatttttcaagaggcaggaagcaggtggtggtcagaaagggatgcctacccttgtgtgacttgtgacagtgactgcttggaagagcgctgggagggtgaggtaggcagagctaggctctctgctgtgtggtagcatagggcatacggtgatacaggggagaagatatgatacctccaagtgttttccctctgtgtctgtctgagtctcttgttgctaaatgagatgtctacgcaacagctgaaagcatttgctttcccaaggcaaatgtttctccagttgtcaaaggaccagtagcagacttcctgcgaatgcaaatgtttaaagaaggatggtgtggggcgttttttgaaaaaaaaaataattctgatttctggtcaggaattaaaaggcagaaagg

SEQ ID NO: 10 - DNA, 2838 bp, Rattus norvegicus
  misc_feature (2265)..(2265): n=a, c, g or t
atgagggcccggagcggggcgcggggcgcgctgctgctggcgctgctgctctgctgggatccgacaccgagcttagcaggcattgactctggtggccaggcactcccagactccttcccatcagcacccgcggagcagctgcctcacttcctgctggaaccagaggatgcctacatcgtaaagaacaagccagtggaattgcactgccgagccttccctgccacacagatctacttcaagtgtaatggcgagtgggttagccagaaaggccacgtcacgcaggagagcctggatgaggccacaggcttgcgaatacgagaggtgcagatagaggtgtcgcggcagcaggtggaggaactttttgggctcgaggactactggtgtcagtgcgtggcctggagctcttcgggaaccaccaagagtcgccgagcctacatccgcattgcctacttgcgcaagaactttgaccaggagcctctggcgaaggaggtacccttggatcatgaggtccttctgcagtgccgcccaccagagggagtgcctgtggctgaggtggaatggctcaagaatgaagatgtcatcgatcccgctcaggacactaacttcctgctcaccattgaccacaacctcatcatccgccaggcgcgcctctcagacacagccaactacacctgtgtggcaaagaatattgtggccaagcgccggagcacgacggccacagtcatcgtctatgtgaacggaggttggtccagctgggcagaatggtcaccctgctctaaccgctgcggccgaggttggcagaaacgtactaggacctgcaccaacccagccccactcaatggaggtgccttctgcgagggacaggcttgccagaagacggcttgcaccaccgtgtgcccagtggatggagcgtggactgagtggagcaagtggtccgcctgcagcacagagtgtgcgcactggcgcagccgcgagtgcatggcaccgccgccccagaacggaggccgtgactgcagcgggacgctacttgactccaagaactgcaccgatgggctgtgcgtgctgaatcagagaactctaaacgaccctaaaagccgccccctggagccgtcgggagacgtggcgctgtatgcgggcctcgtggtggccgtctttgtggttctggcagttctcatggctgtaggagtgatcgtgtaccggagaaactgccgggacttcgacacggacatcactgactcctctgctgccctcactggtggtttccaccccgtcaacttcaagactgcaaggcccagcaacccacagctcctgcacccatccgcccctccggacctaacggccagtgctggcatctaccgcggacctgtgtatgccctgcaggactctgccgacaagatccctatgactaattcaccccttctggatcccttgcccagcctcaagatcaaggtctatgactccagcaccatcggctctggggctggcctggctgatggagccgacctgctgggtgtcttaccacccggtacatacccaggcgatttctcccgggacacccacttcctgcacctgcgcagcgccagccttggttcccagcacctcctgggcctccctcgagaccccagcagcagtgtcagtggcacctttggttgcctgggtgggaggctgaccattcccggcacaggggtcagcctgttggtaccaaatggagccattccccagggcaagttctatgacttgtatctacgtatcaacaagactgaaagcaccctcccactttcggaaggttcccagacagtattgagcccctcggtgacctgcgggcccacgggcctcctcctgtgccgccctgttgtcctcactgtgccccactgtgctgaagtcattgccggagactggatcttccagctcaagacccaggcccatcagggccactgggaggaggtggtgactttggatgaggagactctgaacaccccctgctactgccagctagaggctaaatcctgccacatcctgttggaccagctgggtacctacgtgttcacgggcgagtcctactcccgctccgcagtcaagcggctccagctagccatcttcgccccagccctctgcacctccctggagtatagtctcagggtctactgtctggaggacactcctgcagcactgaaggaggtcctagagctggagaggactctgggtggctacttggtggaggagcccaagactttgctctttaaggacagttaccacaacctacgnctctccctccatgacatcccccatgcccactggaggagcaaactactggccaagtaccaggagattcccttctaccatgtgtggaacggcagccagaaagccctgcactgcactttcaccctggagagacatagcctagcctccactgagttcacctgtaaggtctgcgtgcggcaggtagaaggggaaggccagattttccagctgcacaccacgctggctgagacgcctgctggctccctggatgcactctgctctgcccctggcaatgctgccaccacacagctgggaccctatgccttcaagataccactgtccatccgccagaagatctgcaacagcctggacgcccccaactcacggggcaatgactggcggctgttggcacagaagctctccatggaccggtacctgaactacttcgccaccaaagctagtcccacaggcgtgatcttagacctctgggaagctcggcagcaggatgatggggacctcaacagcctggccagtgccttggaggagatgggcaagagtgagatgctggtagccatgaccactgatggcgattgctga

SEQ ID NO: 11 - DNA, 511 bp, Rattus norvegicus
gcgcccagagcagagcacccgctgcgcagagaccacagcccgcccgccatgatgctaggtaacaaacgaatggggctgtgtggactgaccctcgctctatccctgctcgtgtgtttgggcattctggctgaggggtacccctccaagccggacaatccgggcgaggacgcgccagcagaggacatggccagatactactccgctctgcgacactacatcaatctcatcaccagacagagatatggcaagagatccagccctgagacactgatttcagatctcttaatgagagaaagcacagaaaatgcccccagaacaaggcttgaagacccttccatgtggtgatgggaaatgaaacttgctctcctgacttttcctagtttccccccacatctcatctcatcctgtgaaaccagtctgcctgtcccaccaatgcatgccaccaccaggctggattccgacccatttcccttgttgtcgttgtatatatgtgtgtttaaataaagtatca

SEQ ID NO: 12 - DNA, 2556 bp, Rattus norvegicus
ctttcatactccagccacggaacggagccagggcagacgggtccggattttccccctgccccgaccctcctctccacctcccgccgtcgtgacaccggctgggggcgacaggaggcactgggtacccagaacgaggattgcgagcgttctctgaccatttgcacgaccccagagattgccacatctttcttgttctctgctaaacgtttctcttcggtctctggcagcccgttggtcatgaaaaccttcacgttgccagcatccgtcctcttctgcttccttctactcatccgggggttgggagcagcaccccccgggcgctccgatgtttatcctcctcccctcggctctgagcataatgggcaggtagctgaggacgcagtgtcccggccaaaggatgacagcgtcccagaggtccgagcggctcggaattccgagcctcaggaccagggagagctcttccagggcgtggatccccgggcgctggccgcggtactgttgcaggcactggaccgtccggcctcgcccccggctgtcccggcaggttcccagcagggaacacccgaagaagcagcagaagctctgctgaccgagtccgtgcgcagtcagacccatagcctcccggcatcagaaatccaagcgtccgctgtggcgccccctcgccctcagactcaggacaacgatcccgaggcagacgaccgctcagaagagctggaggcactagcatccttgctccaagaacttcgagatttcagtccgagtaatgctaagcgccagcaagagacggcggcagcagagactgaaacccgcacgcacacgctgacccgagtcaatctggagagccccgggccagagcgcgtatggcgcgcttcctggggagagttccaggcgcgcgtcccggagcgtgctcctctgccgccctcggtcccttctcaattccaggctcgaatgtccgaaaacgttccccttcccgaaacccatcagttcggggaaggagtgtcctcccctaaaacacatcttggtgagactttgacacccttatccaaggcgtaccaaagtctaagtgcccccttccccaaggtgcgtcggctcgagggctcattcctgggcggttccgaggcaggagagcgcctgcttcaacaagggttagctcaggtagaggcagggaggaggcaggcggaggccacccggcaggccgcagcgcaagaagagcggctggccgatctcgcctccgacctgctgctccagtatttgctgcagggcggcgcccggcagcgcgatctcgggggtcgcgggctgcaggagacgcagcaagagcgggagaacgagagggaggaggaggcggagcaggagagacgcggtggtggggaggacgaggtgggggaagaggatgaggaggcggcagaggcggaggcggaggcagaggaggcggagagggcgcggcagaacgcgctcctgttcgccgaggaggaggacggggaagccggagccgaggacaagcgctcccaggaggaggcgccaggccatcggcggaaggatgctgaggggacagaggagggcggggaggaggatgacgacgacgaagagatggatccgcagacgatcgatagtctcattgaactgtccaccaaactccacctgccagcagacgatgtggtcagcatcatcgaagaggtggaggagaaacggaagcggaagaagaacgcccctcccgagccggtgccgccccccagggctgccccagccccgacccatgtccgctccccgcagcccccacctcccgccccggcccgggatgagttgccggactggaacgaagtactcccaccctgggatcgggaggaggatgaggtgtttcccccggggccctatcaccccttcccaaactacattcggccgcggacactgcagccgcccgcatcctcccgccgccgtcacttccatcacgcgttgccacctgcgcgccaccatcccgatctggaggcccaggccaggcgcgcgcaggaggaagcggacgcggaggagcgccggctgcaggagcaggaggagctggagaattacattgagcacgtgctgctgcaccgcccgtgacccgcccctgcgcgcccgctcccaactgcgcgcgccgccacgccccccctccgtgtcgctcctcctccctctcggtgtttgcatgcgccccggctccgcccctcggctgccgcccggccccgccccacaaggccccgccccgggttctgtcaggaccagacctgtcagacttctttggggtctgatcctggggccagcccaggcgggtgtgtggtttgtgcgagtccccttacacccccacttcctccaggggcctcgtccccatctagtttctctagcgacttcctggtcccaaacggggaaaagctgttctatttaatcgtgtgaagtgtctgtctcccagccttggggcccccggagcctcccttctccaaattgctgtgaacttacccacatcttgcccttctgttgtaaatacccctcacggaggaaatagttttgctaagaaataaaagtgactatttt

SEQ ID NO: 13 - DNA, 4127 bp, Rattus norvegicus
agccgctgctggggaggttggggctgaggtggtggcgggcgacgggcctcgagacgcggagcgacgcggcctagcgcggcggacggccgagggaactcgggcagtcgtcccgtcccgccatggaaatggagaaggaattcgagcagatcgataaggctgggaactgggcggctatttaccaggatattcgacatgaagccagtgacttcccatgcagaatagcgaaacttcctaagaacaaaaaccggaacaggtaccgagatgtcagcccttttgaccacagtcggattaaattgcatcaggaagataatgactatatcaatgccagcttgataaaaatggaggaagcccagaggagctatatcctcacccagggccctttaccaaacacgtgcgggcacttctgggagatggtgtgggagcagaagagcaggggcgtggtcatgctcaaccgcatcatggagaaaggctcgttaaaatgtgcccagtattggccacagaaagaagaaaaagagatggtcttcgatgacaccaatttgaagctgacactgatctctgaagatgtcaagtcatattacacagtacggcagttggagttggagaacctggctacccaggaggctcgagagatcctgcatttccactacaccacctggcctgactttggagtccctgagtcacctgcctctttcctcaatttcctattcaaagtccgagagtcaggctcactcagcccagagcacggccccattgtggtccactgcagtgctggcattggcaggtcagggaccttctgcctggctgacacctgcctcttactgatggacaagaggaaagacccgtcctctgtggacatcaagaaagtgctgttggagatgcgcaggttccgcatggggctcatccagacggccgaccaactgcgcttctcctacctggctgtgatcgagggtgcaaagttcatcatgggcgactcgtcagtgcaggatcagtggaaggagctttcccatgaagacctggagcctccccctgagcacgtgcccccacctccccggccacccaaacgcacattggagcctcacaatggcaagtgcaaggagctcttctccaaccaccagtgggtgagcgaggagagctgtgaggatgaggacatcctggccagagaggaaagcagagccccctcaattgctgtgcacagcatgagcagtatgagtcaagacactgaagttaggaaacggatggtgggtggaggtcttcaaagtgctcaggcatctgtccccactgaggaagagctgtccccaaccgaggaggaacaaaaggcacacaggccagttcactggaagcccttcctggtcaacgtgtgcatggccacggccctggcgactggcgcgtacctctgttaccgggtatgttttcactgacagactgctgtgaggcatgagcgtggtgggcgctgccactgcccaggttaggatttggtctgcggcgtctaacctggtgtagaagaaacaacagcttacaagcctgtggtggaactggaagggccagccccaggaggggcatctgtgcactgggctttgaaggagcccctggtcccaagaacagagtctaatctcagggccttaacctgttcaggagaagtagaggaaatgccaaatactcttcttgctctcacctcactcctcccctttctctggttcgtttgtttttggaaaaaaaaaaaaaagaattacaacacattgttgtttttaacatttataaaggcaggtttttgttatttttagagaaaacaaaagatgctaggcactggtgagattctcttgtgccctttggcatgtgatcagattcacgatttacgtttatttccgggggagggtcccacctgtcaggactgtaaagttcctgctggcttggtcagcccccccacccccccaccccgagcttgcaggtgccctgctgtgaggagagcagcagcagaggctgcccctggacagaagcccagctctgcttccctcaggtgtccctgcgtttccatcctccttctttgtgaccgccatcttgcagatgacccagtcctcagcaccccacccctgcagatgggtttctccgagggcctgcctcagggtcatcagaggttggctgccagcttagagctggggcttccatttgattggaaagtcattactattctatgtagaagccactccactgaggtgtaaagcaagactcataaaggaggagccttggtgtcatggaagtcactccgcgcgcaggacctgtaacaacctctgaaacactcagtcctgctgcagtgacgtccttgaaggcatcagacagatgatttgcagactgccaagacttgtcctgagccgtgatttttagagtctggactcatgaaacaccgccgagcgcttactgtgcagcctctgatgctggttggctgaggctgcggggaggtggacactgtgggtgcatccagtgcagttgcttttgtgcagttgggtccagcagcacagcccgcactccagcctcagctgcaggccacagtggccatggaggccgccagagcgagctggggtggatgcttgttcacttggagcagccttcccaggacgtgcagctcccttcctgctttgtccttctgcttccttccctggagtagcaagcccacgagcaatcgtgaggggtgtgagggagctgcagaggcatcagagtggcctgcagcggcgtgaggccccttcccctccgacacccccctccagaggagccgctccactgttatttattcactttgcccacagacacccctgagtgagcacaccctgaaactgaccgtgtaaggtgtcagcctgcacccaggaccgtcaggtgcagcaccgggtcagtcctagggttgaggtaggactgacacagccactgtgtggctggtgctggggcaggggcaggagctgagggtcttagaagcaatcttcaggaacagacaacagtggtgacatgtaaagtccctgtggctactgatgacatgtgtaggatgaaggctggcctttctcccatgactttctagatcccgttccccgtctgctttccctgtgagttagaaaacacacaggctcctgtcctggtggtgccgtgtgcttgacatgggaaacttagatgcctgctcactggcgggcacctcggcatcgccaccactcagagtgagagcagtgctgtccagtgccgaggccgcctgactcccggcaggactcttcaggctctggcctgccccagcacaccccgctggatctcagacattccacacccacacctcattccctggacacttgggcaagcaggcccgcccttccacctctggggtcagcccctccattccgagttcacactgctctggagcaggccaggaccggaagcaaggcagctggtgaggagcaccctcctgggaacagtgtaggtgacagtcctgagagtcagcttgctagcgctgctggcaccagtcaccttgctcagaagtgtgtggctcttgaggctgaagagactgatgatggtgctcatgactcttctgtgaggggaacttgaccttcacattgggtggctttttttaaaataagcgaaggcagctggaactccagtctgcctcttgccagcacttcacattttgcctttcacccagagaagccagcacagagccactggggaaggcgatggccttgcctgcacaggctgaggagatggctcagccggcgtccaggctgtgtctggagcagggggtgcacagcagcctcacaggtgggggcctcagagcaggcgctgccctgtcccctgccccgctggaggcagcaaagctgctgcatgccttaagtcaatacttactcagcagggcgctctcgttctctctctctctctctctctctctctctctctctctctctctctctctaaatggccatagaataaaccattttacaaaaataaaagccaacaacaaagtgctctggaatagcacctttgcaggagcggggggtgtctcagggtcttctgtgacctcaccgaactgtccgactgcaccgtttccaacttgtgtctcactaatgggtctgcattagttgcaacaataaatgtttttaaagaac

SEQ ID NO: 14 - protein, 4 aa, Artificial Sequence
  Synthetic peptide
TLQP

SEQ ID NO: 15 - protein, 4 aa, Artificial Sequence
  Synthetic peptide
LEGS

SEQ ID NO: 16 - protein, 4 aa, Artificial Sequence
  Synthetic peptide
AQEE